SEQUENCE LISTING (13 sequences)

SEQ ID NO: 1 - DNA, 459 bp, synthetic construct
  source 1..459: mol_type = other DNA
caaactgcgcaactcgtgaaaggtaggcggatccccttcgaaggaaagacctgatgcttttcgtgcgcgcataaaataccttgatactgtgccggatgaaagcggttcgcgacgagtagatgcaattatggtttctccgccaagaatctctttgcatttatcaagtgtttccttcattgatattccgagagcatcaatatgcaatgctgttgggatggcaatttttacgcctgttttgctttgctcgacataaagatatccatctacgatatcagaccacttcatttcgcataaatcaccaactcgttgcccggtaacaacagccagttccattgcaagtctgagccaacatggtgatgattctgctgcttgataaattttcaggtattcgtcagccgtaagtcttgatctccttacctctgattttgctgcgcgagtggcagcgacatggtttgttgt

SEQ ID NO: 2 - DNA, 349 bp, synthetic construct
  source 1..349: mol_type = other DNA
cctggccagaatgcaataacgggaggcgctgtggctgatttcgataacctgttcgatgctgccattgcccgcgccgatgaaacgatacgcgggtacatgggaacgtcagccaccattacatccggtgagcagtcaggtgcggtgatacgtggtgtttttgatgaccctgaaaatatcagctatgccggacagggcgtgcgcgttgaaggctccagcccgtccctgtttgtccggactgatgaggtgcggcagctgcggcgtggagacacgctgaccatcggtgaggaaaatttctgggtagatcgggtttcgccggatgatggcggaagttgtcatctctggcttggac

SEQ ID NO: 3 - DNA, 198 bp, synthetic construct
  source 1..198: mol_type = other DNA
  variation 23..34: note = N =A, C, G, or T
aaggcggagagtcagttcgcggnnnnnnnnnnnncggcgcaacgtcgccagctgtctgcacaggagaaatccctgctggcgcataaagatgagacgctggagtacaaacgccagctggctgcacttggcgacaaggttacgtatcaggagcgcctgaacgcgctggcgcagcaggcggataaattcgcacagcagcaa

SEQ ID NO: 4 - DNA, 150 bp, synthetic construct
  source 1..150: mol_type = other DNA
  variation 139..150: note = N =A, G, C, or T
gcgtccactgcatgttatgccgcgttcgccaggcttgctgtaccatgtgcgctgattcttgcgctcaatacgttgcaggttgctttcaatctgtttgtggtattcagccagcactgtaaggtctatcggatttagtgcnnnnnnnnnnnn

SEQ ID NO: 5 - DNA, 100 bp, synthetic construct
  source 1..100: mol_type = other DNA
  variation 89..100: note = N =A, G, C, or T
tcgttagtttctccggtggcaggacgtcagcatatttgctctggctaatggagcaaaagcgacgggcaggtaaagacgtgcattacgtnnnnnnnnnnnn

SEQ ID NO: 6 - DNA, 75 bp, synthetic construct
  source 1..75: mol_type = other DNA
  variation 64..75: note = N =A, G, C, or T
tcgtatcgcatttattgacccggcaaacgggaatgaaacgccgatgtttgtggcgcagggcaannnnnnnnnnnn

SEQ ID NO: 7 - DNA, 51 bp, synthetic construct
  source 1..51: mol_type = other DNA
  variation 40..51: note = N =A, G, C, or T
accgcttcccggtgccgttcacttcccgaataacccggannnnnnnnnnnn

SEQ ID NO: 8 - DNA, 30 bp, synthetic construct
  source 1..30: mol_type = other DNA
  variation 24..30: note = N =A, G, C, or T
acgcggtgacgactatcaggaaannnnnnn

SEQ ID NO: 9 - DNA, 75 bp, synthetic construct
  source 1..75: mol_type = other DNA
  variation 25..41: note = N =A, G, C, or T
  variation 34..41: note = barcode sequence
caagcagaagacggcatacgagatnnnnnnnnnnnnnnnnngtgactggagttcagacgtgtgctcttccgatct

SEQ ID NO: 10 - DNA, 70 bp, synthetic construct
  source 1..70: mol_type = other DNA
  variation 30..37: note = N =A, G, C, or T
  variation 30..37: note = barcode sequence
aatgatacggcgaccaccgagatctacacnnnnnnnnacactctttccctacacgacgctcttccgatct

SEQ ID NO: 11 - DNA, 21 bp, synthetic construct
  source 1..21: mol_type = other DNA
caagcagaagacggcatacga

SEQ ID NO: 12 - DNA, 33 bp, synthetic construct
  source 1..33: mol_type = other DNA
agatcggaagagcacacgtctgaactccagtca

SEQ ID NO: 13 - DNA, 33 bp, synthetic construct
  source 1..33: mol_type = other DNA
agatcggaagagcgtcgtgtagggaaagagtgt

(19) United States
(12) Patent Application Publication
(10) Pub. No.: US 2026/0078439 A1
(43) Pub. Date: Mar. 19, 2026

(54) Next-Generation Sequencing Pipeline for Detection of Ultrashort Single-Stranded Cell-Free DNA

(71) Applicant: THE REGENTS OF THE UNIVERSITY OF CALIFORNIA, Oakland, CA (US)

(72) Inventors: Jordan Cheng, Oakland, CA (US); David Wong, Beverly Hills, CA (US); Feng Li, Northridge, CA (US); Neeti Swarup, Oakland, CA (US)

(21) Appl. No.: 19/106,154

(22) PCT Filed: Aug. 24, 2023
(86) PCT No.: PCT/US2023/072792

§ 371 Date: Feb. 24, 2025

Related U.S. Application Data
(60) Provisional application No. 63/373,369, filed on Aug. 24, 2022.

(51) Int. Cl. C12Q 1/6869; C12N 15/10; C12Q 1/37; C12Q 1/6806; C12Q 1/6809

(52) U.S. Cl. CPC C12Q 1/6869; C12N 15/1013; C12Q 1/37; C12Q 1/6806; C12Q 1/6809; G01N 2333/96441

(57) ABSTRACT

A method of isolating ultrashort single-stranded cell-free DNA (uscfDNA) is described as well as methods of using the uscfDNA for detecting biomarkers and diagnosing diseases and disorders.

### CROSS REFERENCE TO RELATED APPLICATIONS

This application claims priority to U.S. Provisional Application No. 63/373,369, filed Aug. 24, 2022, which is hereby incorporated by reference herein in its entirety.

### STATEMENT OF GOVERNMENT SUPPORT

This invention was made with government support under Grant Number CA233370, CA264398 and DE031531, awarded by the National Institutes of Health. The government has certain rights in the invention.

### REFERENCE TO AN EXTENSIBLE MARKUP LANGUAGE (XML) SEQUENCE LISTING

The present application hereby incorporates by reference the entire contents of the sequence listing as submitted in the XML file named “206030-0269-00WO_SequenceListing.xml” in XML format, which was created on Aug. 22, 2023, and is 17,827 bytes in size.

### BACKGROUND OF THE INVENTION

In liquid biopsy, cell-free DNA (cfDNA) analysis is typically focused on the mono-nucleosomal cfDNA (mncfDNA) biomarker of approximately 160 bp in length. However, the current impression of the average fragment length of cfDNA is influenced by the inherent biases of nucleic acid extraction and library preparation. The recent adoption of single-stranded library preparation methods for cfDNA analysis suggests that in addition to mncfDNA, there are shorter cfDNA fragments (<100 bp) that can originate from either single-stranded or nicked dsDNA in plasma (Burnham et al., Sci Rep, 2016, 6; Snyder et al., Cell, 2016, (164) 57-68). Previous studies indicate that size-selecting for shorter fragments of cfDNA will enrich for mutant-containing cfDNA fragments in late-stage cancer patients (Mouliere and Rosenfeld, Proc Natl Acad Sci, 2015, (112) 3178-3179). Next-generation sequencing approaches examining whole-genome differences in plasma cfDNA fragment lengths have revealed distinct fragment-profiles in cancer patients compared to those of healthy donors (Cristiano et al., Nature, 2019, (570) 385-389). Additionally, groups have attempted to utilize cfDNA strandedness as a diagnostic indicator (Huang et al., Pathol. Oncol. Res, 2020, (26) 2621-2632; Zhu et al., Mol Diagn Ther, 2020, (24) 95-101). With these considerations, ultrashort single-stranded cell-free DNA (uscfDNA) is an unexamined cfDNA entity with potential clinical relevance. In general, nucleic acid extraction kits are not designed to efficiently retain low-molecular cfDNA (<100 bp) regardless of strandedness (Diefenbach et al., Cancer Genet, 2018, 228-229, 21-27).

Thus, there remains a need in the art for an effective ultrashort ssDNA cfDNA extraction method which retains low-molecular ultrashort cfDNA as well as efficient single-stranded library preparation methods. This invention stratifies the unmet needs.

### SUMMARY OF THE INVENTION

In one embodiment, the invention relates to a method of isolating ultrashort single-stranded cell-free DNA (uscfDNA) molecules from a sample, the method comprising the steps of: a) contacting the sample with Solid Phase Reversible Immobilization (SPRI) magnetic beads to capture the uscfDNA; b) contacting the sample with a mixture of phenol:chloroform:isoamyl alcohol to separate the uscfDNA away from contaminating proteins and peptides; c) contacting the sample with Solid Phase Reversible Immobilization (SPRI) magnetic beads to clean up the uscfDNA; and d) extraction of the uscfDNA. In one embodiment, the method further comprises the step of preparing a sequencing library from the extracted uscfDNA.

In one embodiment, the method further comprises the step of sequencing the library of uscfDNA. In one embodiment, the method further comprises the step of lysing a cell or disrupting proteins prior to step a).

In one embodiment, the step of lysing a cell or disrupting proteins comprises: i) adding Proteinase K and SDS to the sample, ii) incubating the sample for 30 minutes at 60° C., and iii) cooling the sample to ambient room temperature. In one embodiment, step a) comprises: i) adding SPRI magnetic size selection beads and isopropanol to the sample, ii) incubating the sample at room temperature for at least 10 minutes, iii) centrifuging the sample at 4000×G for at least five minutes, iv) removing and discarding the supernatant, and v) resuspending the pellet in buffer.

In one embodiment, step b) comprises: i) aliquoting the resuspension solution from step a) v) into phase lock tubes, ii) adding an equal volume (to the aliquot of the resuspension solution) of phenol:chloroform:isoamyl alcohol with equilibrium buffer, iii) vortexing for at least 15 seconds, iv) centrifuging the tubes at 19000×G for at least five minutes, v) transferring the upper clear supernatant to a new tube; and vi) repeating steps ii)-v) twice.

In one embodiment, step c) comprises performing at least two rounds of SPRI bead based clean up followed by ethanol precipitation.

In one embodiment, the sample is a biological fluid sample. In one embodiment, the sample is a blood sample, a plasma sample, a saliva sample, a sputum sample, a urine sample or a liquid biopsy sample.

In one embodiment, the invention relates to a method of identifying novel biomarkers for diseases or disorders comprising obtaining uscfDNA from a sample according to the method of any one of claims 1-10 and analyzing the amount or sequence content of the uscfDNA to identify novel biomarkers of a disease or disorder. In one embodiment, the biomarker is selected from the group consisting of a mutation, an indel, a copy number variation, and a methylation marker. In one embodiment, the biomarker is an increase or decrease in the total amount of uscfDNA in a test sample as compared to a control sample. In one embodiment, the biomarker is an increase or decrease in the amount of uscfDNA associated with a specific gene in a test sample as compared to a control sample.

In one embodiment, the invention relates to a method of diagnosing a diseases or disorder in a subject in need thereof, the method comprising obtaining a sample from the subject, isolating uscfDNA from the sample using the uscfDNA isolation method comprising the steps of: a) contacting the sample with Solid Phase Reversible Immobilization (SPRI) magnetic beads to capture the uscfDNA; b) contacting the sample with a mixture of phenol:chloroform:isoamyl alcohol to separate the uscfDNA away from contaminating proteins and peptides; c) contacting the sample with Solid Phase Reversible Immobilization (SPRI) magnetic beads to clean up the uscfDNA; d) extraction of the uscfDNA; e) preparing a sequencing library from the extracted uscfDNA; and e) sequencing the library of uscfDNA; analyzing the amount or sequence content of the uscfDNA to detect a biomarker of a disease or disorder, and diagnosing the subject as having or at risk of the disease or disorder associated with the identified biomarker. In one embodiment, the biomarker is a mutation, an indel, a copy number variation, or a methylation marker. In one embodiment, the biomarker is an increase or decrease in the total amount of uscfDNA in a test sample as compared to a control sample. In one embodiment, the biomarker is an increase or decrease in the amount of uscfDNA associated with a specific gene in a test sample as compared to a control sample.

In one embodiment, the disease or disorder is selected from the group consisting of an autoimmune disease or disorder, a disease or disorder associated with an infectious agent, and cancer. In some embodiments, the method further includes a step of administering a treatment for the diagnosed disease or disorder.

In one embodiment, the invention relates to a kit comprising components and reagents for isolating uscfDNA from the sample using the uscfDNA isolation method comprising the steps of: a) contacting the sample with Solid Phase Reversible Immobilization (SPRI) magnetic beads to capture the uscfDNA; b) contacting the sample with a mixture of phenol:chloroform:isoamyl alcohol to separate the uscfDNA away from contaminating proteins and peptides; c) contacting the sample with Solid Phase Reversible Immobilization (SPRI) magnetic beads to clean up the uscfDNA; d) extraction of the uscfDNA. In some embodiments the kit further includes components or reagents for preparing a sequencing library from the extracted uscfDNA.

### BRIEF DESCRIPTION OF THE DRAWINGS

FIG. 1A and FIG. 1B depict representative schematic diagrams of the Broad-Range Cell-Free DNA Sequencing (BRcfDNA-Seq). FIG. 1A depicts a representative schematic diagram of three different extraction protocols, QiaC, referring to the QIAGEN QIAamp Circulating Nucleic Acid Kit regular protocol, QiaM, referring to the miRNA protocol of the QIAamp Circulating Nucleic Acid Kit, and SPRI, referring to the Solid Phase Reversible Immobilization magnetic beads and phenol:chloroform:isoamyl alcohol protocol. Compared to QiaC, QiaM and SPRI protocols utilize an increased ratio of isopropanol in order to retain the low-molecular weight nucleic acids for downstream analysis. FIG. 1B depicts a representative schematic diagram of single-stranded library preparation, which can incorporate dsDNA, ssDNA, and nicked DNA into the library. Unique molecular identifiers (UMI) are incorporated during the library preparation to remove PCR duplicates.

FIG. 2A through FIG. 2F depicts representative populations of ultrashort cfDNA fragments in the plasma of healthy donors. FIG. 2A depicts a representative image of an electropherogram of BRcfDNA-Seq using QiaM or PSPRI, revealing a distinct final NGS library uscfDNA band at 200 bp (˜50 bp after adapter dimer subtraction) compared to QiaC, cropped for representative sizes. FIG. 2B depicts representative quantification of data from the data depicted in FIG. 2A. QiaM and SPRI extraction methods can reproducibly isolate the 200 bp fragment (180-250 bp region in the electropherogram) in ten human donors based on quantification of electrophoresis output (200 bp band intensity divided by (200 bp+300 bp (250-350 bp region))—bands are elongated with ˜150 bp of adapters on both sides). ***, p<0.001. The paired two-tailed Student's T-test was performed after ANOVA analysis. Average±S.E.M. See also FIG. 4. FIG. 2C depicts a representative alignment of total mapped reads from QiaC, QiaM, and SPRI extraction, demonstrating that only QiaM and SPRI extracted samples show the native uscfDNA at 50 bp in addition to the mncfDNA peak at ˜160 bp observed in all three samples when adapters are trimmed. Gray line represents sequencing of no template control. FIG. 2D depicts representative chromosomal coverage along the genome by uscfDNA of QiaC, QiaM, and SPRI. See also FIG. 6. FIG. 2E depicts a representative heatmap of correlation (Pearson) between uscfDNA and mncfDNA coverage of 100 bp genome bins for each of the three methods, revealing similarity between the mappings of uscfDNA and mncfDNA groups. FIG. 2F depicts representative functional group analysis of the reads of mncfDNA and uscfDNA, showing that uscfDNA is more similar to the genomic profile. Different extraction methods alter the proportion of functional elements. See also FIGS. 3 and 4.

FIG. 3A through FIG. 3C depict representative imaging of QiaM results relative to QiaC. FIG. 3A depicts a representative electropherogram demonstrating that the increased isopropanol (1.8 mL to 2.3 mL) is integral to retaining the uscfDNA from plasma. FIG. 3B depicts representative SEM images of a Qiagen silica filter showing sheet-like deposits (black arrows) only in QiaM extraction of plasma. Scale bars represent 50 μm. FIG. 3C depicts a representative electropherogram demonstrating the recovery of uscfDNA from a QiaC plasma extraction. Centrifugation, rather than a vacuum, was used so that the flow-through could be collected, which was subsequently extracted with QiaM to reveal the rescue of the uscfDNA band.

FIG. 4A through FIG. 4D depict representative electropherograms confirming that uscfDNA is consistently observed. FIG. 4A depicts representative electropherogram images of ten healthy donors when samples were extracted with QiaC, QiaM, and SPRI, showing the presence of uscfDNA. FIG. 4B depicts representative electropherograms demonstrating uscfDNA exists independently of the whole blood collection tube. FIG. 4C depicts representative quantification of nucleotides from a TE buffer control extracted with all three methods, demonstrating that uscfDNA or mncfDNA peaks are not produced when aligned with the human genome. FIG. 4D depicts a representative electropherogram of RNase cocktail digestion prior to library preparation, demonstrating RNase does not reduce the uscfDNA band in QiaM and SPRI extracted samples.

FIG. 5A and FIG. 5B depict representative data demonstrating magnetic bead extraction methods capture short and single-stranded DNA molecules better than silica column-based methods. FIG. 5A depicts a representative electropherogram of the extraction of healthy plasma spiked with a ladder of short lambda ssDNA oligos, demonstrating various retention efficiencies between QiaC, QiaM, and SPRI methods. FIG. 5B depicts representative quantification after alignment to the lambda genome, showing QiaM and SPRI methods have greater efficiency of extracting ultrashort ssDNA molecules.

FIG. 6A and FIG. 6B, depicts representative quantification of mitochondrial contribution to cfDNA. FIG. 6A depicts representative diagrams demonstrating the majority of DNA aligns to the nuclear genome and not to the mitochondrial genome. Square indicates the visual representation of mitochondria reads. FIG. 6B depicts representative quantification of aligned reads, demonstrating QiaM and SPRI are enriched for mitochondrial DNA in the uscfDNA population but still makes up a minor fraction of total DNA.

FIG. 7A and FIG. 7B, depicts representative single strand and double strand populations of uscfDNA in QiaM and SPRI extraction. FIG. 7A depicts representative size distribution of final library digestion with cfDNA supplemented with control oligos. FIG. 7B depicts representative size distribution of library preparation variation with cfDNA supplemented with control oligos. Top panels: electrophoretic visualization. Middle panels: quantification of the mapped reads belonging to the short (uscfDNA) or long population (mncfDNA). Bottom panels: mapped read size distribution. Reads with insert size under 25 bp and above 250 bp were excluded. Bar graphs composed of plasma from three different human donors. The paired two-tailed Student's T-test was performed after ANOVA analysis. *, p<0.05; **, p<0.01; ***, p<0.001. Sequences from the lambda genome of 460 bp dsDNA and 356 nt ssDNA were used as positive controls. Adapter-dimers have been cropped from the presented electropherograms. Mean±S.E.M. Electropherogram images were cropped for representative sizes. See also FIGS. 8 and S6.

FIG. 8A and FIG. 8B depict representative electropherograms of final libraries prepared from different treatments. FIG. 8A depicts representative electropherograms of final libraries constructed from extracted cfDNA after nuclease digestion. FIG. 8B depicts representative electropherograms of final libraries constructed from extracted cfDNA after undergoing ssDNA library preparation, dsDNA library preparation, and nick-repair enzyme treatment. Replicate experiments using plasma from three healthy donors extracted by QiaM and SPRI.

FIG. 9A and FIG. 9B depict representative fragment length distribution of aligned reads from samples that underwent digestions or variations in the library preparation method. FIG. 9A depicts representative alignment of sequenced libraries to the human genome pretreated by digestions and library preparation variations on a sample from Donor 1 of FIG. 5 extracted by QiaM. FIG. 9B depicts representative alignment of sequenced libraries to the human genome pretreated by digestions and library preparation variations on a sample from Donor 1 of FIG. 5 extracted by SPRI. Reads with insert size under 25 bp and above 250 bp were excluded from the plots.

FIG. 10A through FIG. 10D depict representative heatmap correlation of uscfDNA and mncfDNA reads. FIG. 10A depicts representative heatmap correlation of uscfDNA and mncfDNA reads of various digestions of samples extracted by QiaM. FIG. 10B depicts representative heatmap correlation of uscfDNA and mncfDNA reads of various digestions of samples extracted by SPRI. FIG. 10C depicts representative individual functional element peak analysis of sequenced reads from digestions of QiaM from FIG. 3. FIG. 10D depicts representative individual functional element peak analysis of sequenced reads from digestions of SPRI from FIG. 3. Values are summated in FIG. 4.

FIG. 11A through FIG. 11C depict representative enrichment of mncfDNA or uscfDNA using pre-library digestion to reveal functional characteristics. FIG. 11A depicts a representative function peak profile in mncfDNA and uscfDNA fractions of QiaM extraction after ssDNA enrichment treatments (dsDNase and Heatshock-) and dsDNA enrichment treatments (S1, exo1, and dsLibrary preparation) along different elements of a typical gene. FIG. 11B depicts a representative function peak profile in mncfDNA and uscfDNA fractions of SPRI extraction after ssDNA enrichment treatments (dsDNase and Heatshock-) and dsDNA enrichment treatments (S1, exo1, and dsLibrary preparation) along different elements of a typical gene. FIG. 11C depicts representative quantification of the proportion of functional peaks relative to the genome (grey dotted line) at different uscfDNA fragment sizes. Different patterns are observed in different extraction methods. Bar graphs: Mean±S.E.M. See also FIGS. 10 and 12.

FIG. 12 depicts representative quantification of functional peaks at different fragment sizes. Functional peaks were first called with macs2 (2.2.7.2 version) and then analyzed with HOMERannotatePeaks (version 4.11.1).

FIG. 13 depicts a table of the NGS statistics.

FIG. 14 depicts a Next-generation Sequencing (NGS) pipeline to detect ultrashort single-stranded cell-free DNA (uscfDNA).

### DETAILED DESCRIPTION

The invention is based, in part, on the development of a novel method for isolating ultrashort single-stranded cell-free DNA (uscfDNA) from samples. In some embodiments, the method involves contacting the sample with SPRI beads to retain the uscfDNA and performing a phenol chloroform extraction to separate the uscfDNA from proteins and peptides followed by DNA clean-up in the presence of SPRI beads to retain uscfDNA. In some embodiments, the invention relates to sequencing libraries generated from samples containing or retaining uscfDNA, wherein the sequencing libraries have better coverage of promote and exon regions due to the presence of uscfDNA.

In some embodiments, the invention provides methods of use of samples in which the uscfDNA has been enriched for identification of novel biomarkers or for diagnosing diseases or disorders based on the detection of known biomarkers associated with diseases or disorders.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, an “adaptor” of the present invention means a piece of nucleic acid that is added to a nucleic acid of interest, e.g., the polynucleotide. Two adaptors of the present invention are preferably ligated to the ends of a DNA fragment cross-linked to a polypeptide of interest, with one adaptor on each end of the fragment. Adaptors of the present invention can comprise a primer binding sequence, a random nucleotide sequence, a barcode, or any combination thereof.

An affinity label, as the term us used herein, refers to a moiety that specifically binds another moiety and can be used to isolate or purify the affinity label, and compositions to which it is bound, from a complex mixture. One example of such an affinity label is a member of a specific binding pair (e.g, biotin:avidin, antibody:antigen). The use of affinity labels such as digoxigenin, dinitrophenol or fluorescein, as well as antigenic peptide ‘tags’ such as polyhistidine, FLAG, HA and Myc tags, is envisioned.

“Amplification,” as used herein, refers to any in vitro process for increasing the number of copies of a nucleotide sequence or sequences, i.e., creating an amplification product which may include, by way of example additional target molecules, or target-like molecules or molecules complementary to the target molecule, which molecules are created by virtue of the presence of the target molecule in the sample. These amplification processes include but are not limited to polymerase chain reaction (PCR), multiplex PCR, Rolling Circle PCR, ligase chain reaction (LCR) and the like, in a situation where the target is a nucleic acid, an amplification product can be made enzymatically with DNA or RNA polymerases or transcriptases. Nucleic acid amplification results in the incorporation of nucleotides into DNA or RNA. As used herein, one amplification reaction may consist of many rounds of DNA replication. PCR is an example of a suitable method for DNA amplification. For example, one PCR reaction may consist of 2-40 “cycles” of denaturation and replication.

“Amplification products,” “amplified products” “PCR products” or “amplicons” comprise copies of the target sequence and are generated by hybridization and extension of an amplification primer. This term refers to both single stranded and double stranded amplification primer extension products which contain a copy of the original target sequence, including intermediates of the amplification reaction.

A “barcode”, as used herein, refers to a nucleotide sequence that serves as a means of identification for sequenced polynucleotides of the present invention. Barcodes of the present invention may comprise at least 4, 5, 6, 7, 8, 9, 10, 11, 12, 13, 14, 15, 16, 17, 18, 19, 20 or more bases in length.

“Nucleic acid” or “oligonucleotide” or “polynucleotide” or “nucleic acid fragment” as used herein may mean at least two nucleotides covalently linked together. The depiction of a single strand also defines the sequence of the complementary strand, or the sequence of a molecule that hybridizes to at least a portion of the single strand sequence. Thus, a nucleic acid also encompasses the complementary strand of a depicted single strand as well as probes, primers or oligonucleotide sequences having complementarity to at least a portion of the strand. Many variants of a nucleic acid may be used for the same purpose as a given nucleic acid. Thus, a nucleic acid also encompasses substantially identical nucleic acids and complements thereof. A single strand provides a probe that may hybridize to a target sequence. Thus, a nucleic acid also encompasses a probe that hybridizes under appropriate hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. As used herein, the term nucleic acids includes both natural and non-natural nucleic acids. Non-natural nucleic acids include, but are not limited to, 2′F, 2′-fluoro; 2′OMe, 2′-O-methyl; LNA, locked nucleic acid; FANA, 2′-fluoro arabinose nucleic acid; HNA, hexitol nucleic acid; 2′MOE, 2′-O-methoxyethyl; ribuloNA, (1′-3′)-β-L-ribulo nucleic acid; TNA, α-L-threose nucleic acid; tPhoNA, 3′-2′ phosphonomethyl-threosyl nucleic acid; dXNA, 2′-deoxyxylonucleic acid; PS, phosphorothioate; phNA, alkyl phosphonate nucleic acid; and PNA, peptide nucleic acid.

“Primer” as used herein refers to a single-stranded oligonucleotide or a single-stranded polynucleotide that is extended on its 3′ end by covalent addition of nucleotide monomers during amplification. Nucleic acid amplification often is based on nucleic acid synthesis by a nucleic acid polymerase. Many such polymerases require the presence of a primer that can be extended to initiate such nucleic acid synthesis.

As used herein, “sample” or “test sample,” may refer to any source used to obtain nucleic acids for examination using the compositions and methods of the invention. A test sample is typically anything suspected of containing a target sequence.

Any DNA sample may be used in practicing the present invention, including without limitation eukaryotic, prokaryotic, viral DNA, non-natural DNA, cDNA, and recombinant DNA molecules.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The invention provides assays for capture of ultrashort nucleic acid molecules, methods of use thereof for sequencing library construction and methods of use thereof to identify the quantity or sequence(s) of ultrashort cell free (uscf) nucleic acid molecules in a sample. In some embodiments, the uscf nucleic acid molecules are single stranded DNA molecules.

The present technology provides improved nucleic acid preparation compositions and methods suitable for enrichment, isolation and analysis of ultrashort single stranded nucleic acid species sometimes found in cell free or substantially cell free biological compositions containing mixed compositions, and often associated with various disease conditions or apoptotic cellular events (e.g., cancers and cell proliferative disorders, prenatal or neonatal diseases, genetic abnormalities, and programmed cell death events). The ultrashort single stranded nucleic acid species targets, which can represent degraded or fractionated nucleic acids, can also be used for haplotyping and genotyping analysis, such as fetal genotyping for example.

Methods and compositions described herein are useful for size selection of ultrashort single-stranded cell-free DNA, in a simple, cost effective manner that also can be compatible with automated and high throughput processes and apparatus. Methods and compositions provided herein are useful for enriching or extracting a target nucleic acid from a cell free or substantially cell free biological composition containing a mixture of non-target nucleic acids, based on the size of the nucleic acid, where the target nucleic acid is of a different size, and often is smaller, than the non-target nucleic acid.

Methods for Obtaining and Using uscfDNA

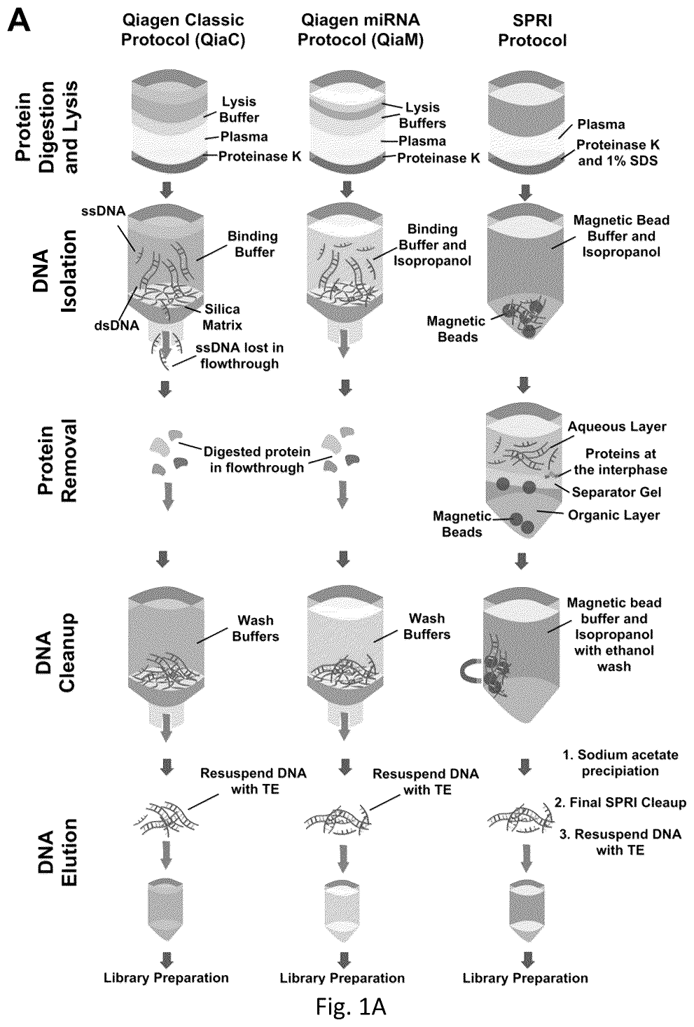

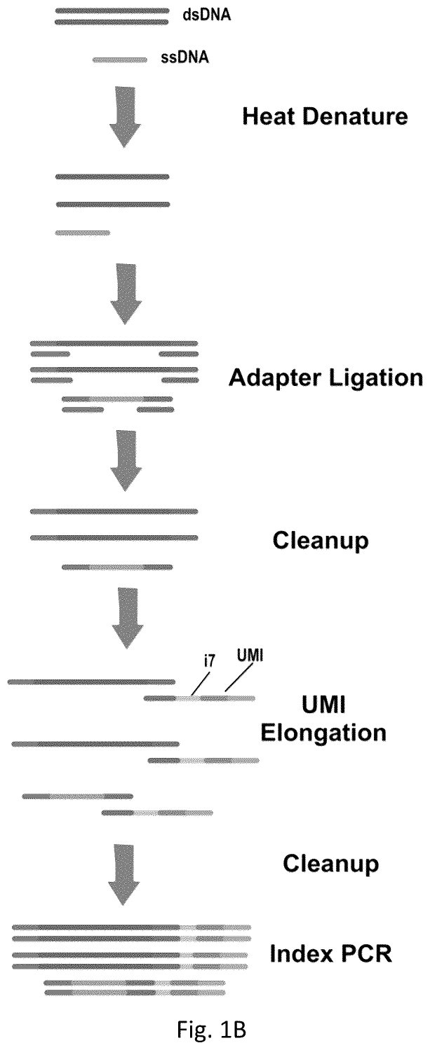

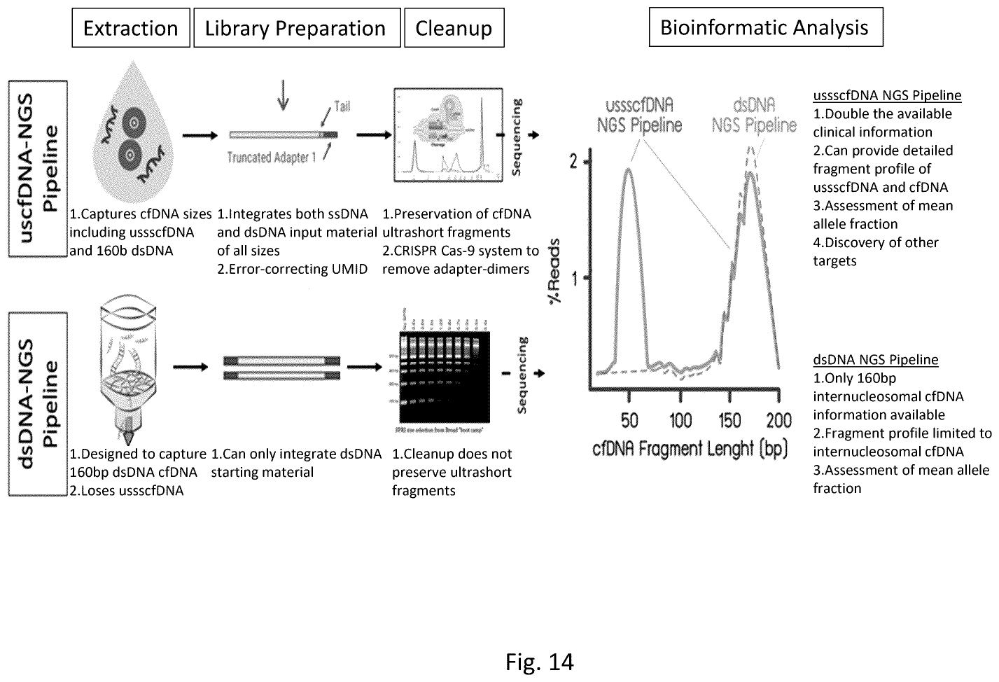

The invention is based, in part on the development of a new pipeline for sequencing uscfDNA. It is represented in FIG. 1A and FIG. 14. While the process is described for sequencing uscfDNA from plasma samples, many of the process steps apply in sequencing uscfDNA found in other types of sample such as urine, sweat, saliva etc. The baseline process may have the following steps: 1) collect a patient sample 2) extract uscfDNA from the sample using an extraction method optimized for uscfDNA, 3) prepare a sequencing library from the extracted uscfDNA and 4) perform next generation sequencing on the sequencing library.

In some embodiments, the extraction method optimized for uscfDNA utilizes Solid Phase Reversible Immobilization (SPRI) magnetic beads and phenol:chloroform:isoamyl alcohol protocol, referred to herein as the SPRI method or SPRI protocol. In some embodiments, the SPRI includes contacting the uscfDNA with at SPRI beads during the DNA isolation step and again during the DNA cleanup step. In some embodiments, the SPRI method includes a phenol chloroform step to separate the uscfDNA from proteins or peptides. In some embodiments, the SPRI method comprises an ordered set of steps as follows: 1) cell lysis and/or protein digestion, 2) SPRI bead-based DNA isolation, 3) a phenol chloroform step to separate the uscfDNA from proteins or peptides, 4) SPRI bead-based DNA clean-up and 5) DNA elution. In some embodiments, the SPRI method further comprises the step of library preparation of the eluted uscfDNA. In some embodiments, the SPRI assay comprises the steps of: adding Proteinase K and SDS to a sample, incubating the sample for 30 minutes at 60° C., cooling the sample to ambient room temperature, adding SPRI magnetic size selection beads and isopropanol to the sample, incubating the sample at room temperature 10 minutes, centrifuging the sample at 4000×G for five minutes, removing and discarding the supernatant, resuspending the pellet in 1×TE Buffer, aliquoting the resuspension solution into phase lock tubes, adding an equal volume (to the aliquot of the resuspension solution) of phenol:chloroform:isoamyl alcohol with equilibrium buffer, vortexing for 15 seconds, centrifuging the tubes at 19000×G for five minutes, repeating the phenol:chloroform:isoamyl alcohol extraction twice (adding phenol:chloroform:isoamyl alcohol, vortexing and centrifuging), transferring the upper clear supernatant to a new tube, adding magnetic SPRI size selection beads and isopropanol to the upper clear supernatant sample, incubating for 10 minutes at room temperature, placing the tube on a magnetic rack for five minutes to allow for the beads to migrate, discarding the supernatant, washing the beads twice with 85% ethanol, removing the ethanol wash and allowing the beads to air dry, resuspending the dried beads in elution buffer, incubating the beads for 2 minutes, contacting the tube with a magnet to separate the beads and allowing the solution to clear, transferring the cleared elution solution to a new tube and adding glycogen, 1×TE Buffer, sodium acetate and 100% ethanol, incubating the solution overnight at −80° C. to precipitate the nucleic acid molecules, centrifuging the tube containing the precipitated nucleic acid molecules at 19000×G for 15 minutes, discarding the supernatant, repeating the ethanol wash step twice with 80% ethanol, removing the supernatant, resuspending the pellet in elution buffer and combining with SPRI and isopropanol and incubating for 10 minutes, placing the tube on a magnetic rack for five minutes to allow for the beads to migrate, discarding the supernatant, washing twice with 80% ethanol, removing the wash and allowing the beads to air dry, and resuspending in elution buffer.

In some embodiments, the methods of the invention include a step of obtaining a plasma fraction of the whole blood sample, wherein the plasma fraction comprises the ultrashort single-stranded cell-free DNA. In some embodiments, the methods of the invention include a step of obtaining saliva sample wherein the saliva sample comprises the ultra-short single-stranded cell-free DNA (uscfDNA).

In some embodiments, the invention relates to a method of isolating uscfDNA from a sample using the miRNA protocol of the QIAamp Circulating Nucleic Acid Kit, referred to herein as the QiaM method.

### Library Preparation

In some embodiments the methods of the invention include the preparation of a sequencing library from the uscfDNA. In some embodiments, the method of the invention includes attaching sequencing adapters to ends of ultrashort single-stranded cell-free DNA fragments, thereby preparing a sequencing library comprising library fragments having the sequencing adapters attached to either end of the ultrashort single-stranded cell-free DNA fragments.

In some embodiments, a low molecular weight retention protocol for preparation of a sequencing library is followed for all bead-clean up steps during sequencing library preparation. In some embodiments, for double-stranded DNA libraries extracted uscfDNA is ligated to adapters using standard methodologies in the art with some modifications: the second (or post-PCR) purification is performed using 60 μl of purification beads in order to retain the uscfDNA fragments. In some embodiments, for double-stranded DNA libraries extracted uscfDNA is used as input and heat-shocked prior to ligation to adapters using a single-stranded library preparation method.

### Multiplex Sequencing

The large number of sequence reads that can be obtained per sequencing run permits the analysis of pooled samples i.e. multiplexing, which maximizes sequencing capacity and reduces workflow. For example, the massively parallel sequencing of eight libraries performed using the eight lane flow cell of the Illumina Genome Analyzer, and Illumina's HiSeq Systems, can be multiplexed to sequence two or more samples in each lane such that 16, 24, 32 etc. or more samples can be sequenced in a single run. Parallelizing sequencing for multiple samples i.e. multiplex sequencing, requires the incorporation of sample-specific index sequences, also known as barcodes, during the preparation of sequencing libraries. Sequencing indexes are distinct base sequences of about 5, about 10, about 15, about 20 about 25, or more bases that are added at the 3′ end of the genomic and marker nucleic acid. The multiplexing system enables sequencing of hundreds of biological samples within a single sequencing run. The preparation of indexed sequencing libraries for sequencing of clonally amplified sequences can be performed by incorporating an index sequence into a PCR primer used for cluster amplification. Alternatively, the index sequence can be incorporated into the adaptor, which is ligated to the uscfDNA prior to the PCR amplification. Sequencing of the uniquely marked indexed nucleic acids provides index sequence information that identifies samples in the pooled sample libraries, and sequence information of marker molecules correlates sequencing information of the genomic nucleic acids to the sample source. In embodiments wherein the multiple samples are sequenced individually i.e. singleplex sequencing, marker and uscfDNA of each sample need only be modified to contain the adaptor sequences as required by the sequencing platform and exclude the indexing sequences.

### Samples

In some embodiments, the sample containing uscfDNA is derived from a biological fluid, cell, tissue, organ, or organism, comprising a nucleic acid or a mixture of nucleic acids comprising at least one uscfDNA molecule. Such samples include, but are not limited to sputum/oral fluid, amniotic fluid, blood, a blood fraction, or fine needle biopsy samples (e.g., surgical biopsy, fine needle biopsy, etc.) urine, peritoneal fluid, pleural fluid, and the like. Although the sample is often taken from a human subject (e.g., patient), the assays can be from any mammal, including, but not limited to, dogs, cats, horses, goats, sheep, cattle, pigs, etc.

The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the uscf nucleic acid(s) of interest remain in the test sample. Such “treated” or “processed” samples are still considered to be biological samples with respect to the methods described herein.

### Applications

Sequence information generated as described herein can be used for any number of applications. Exemplary applications include, but are not limited to, determining mutations, indels, copy number variations (CNVs), identify methylation markers, or identifying biomarkers for diseases or disorders using the uscfDNA. The methods and apparatus described herein may employ next generation sequencing technology (NGS) as described elsewhere herein. In certain embodiments, clonally amplified uscfDNA molecules are sequenced in a massively parallel fashion within a flow cell (e.g. as described in Volkerding et al., 2009, Clin Chem, 55:641-658; Metzker, 2010, Nature Rev, 11:31-46). In addition to high-throughput sequence information, NGS provides quantitative information, in that each sequence read is a countable “sequence tag” representing an individual clonal DNA template or a single DNA molecule. In some embodiments, the methods and apparatus disclosed herein may employ the following some or all of the operations from the following: obtain a nucleic acid test sample 0.5 from a patient (typically by a non-invasive procedure); process the test sample in preparation for sequencing; sequence nucleic acids from the test sample to produce numerous reads (e.g., at least 10,000); align the reads to portions of a reference sequence/genome and determine the amount of DNA (e.g., the number of reads) that map to defined portions the reference sequence (e.g., to defined chromosomes or chromosome segments); calculate a dose of one or o more of the defined portions by normalizing the amount of DNA mapping to the defined portions with an amount of DNA mapping to one or more normalizing chromosomes or chromosome segments selected for the defined portion; determining whether the dose indicates that the defined portion is “affected” (e.g., aneuploidy or mosaic); reporting the determination and optionally converting it to a diagnosis; using the diagnosis or determination to develop a plan of treatment, monitoring, or further testing for the patient. In some embodiments, the biological sample is obtained from a subject and comprises a mixture of nucleic acids contributed by different subjects.

### Diagnostic Assays

In some embodiments, use of the methods described herein in the diagnosis, and/or monitoring, and or treating pathologies is contemplated. For example, the methods can be applied to determining the presence or absence of a disease, to monitoring the progression of a disease and/or the efficacy of a treatment regimen, or to determining the presence or absence of nucleic acids of a pathogen e.g. virus. To date a number of studies have reported biomarkers in genes involved in inflammation and the immune response, infectious disease, neurological and psychiatric diseases, and cancer. Biomarkers associated with these diseases and disorder can be identified in uscfDNA enriched samples generated according to the methods of the invention.

In some embodiments, blood, plasma and serum DNA from cancer patients contains measurable quantities of tumor DNA, that can be identified using the methods of the invention to identify the type or stage of the tumor. Identification of genomic instabilities associated with cancers that can be determined in the circulating uscfDNA in cancer patients is a potential diagnostic and prognostic tool. In one embodiment, methods described herein are used to determine a biomarker, mutation or CNV of one or more sequence(s) of interest in a sample, e.g., a sample comprising a mixture of nucleic acids derived from a subject that is suspected or is known to have cancer. In one embodiment, the sample is a plasma sample derived (processed) from peripheral blood that may comprise a mixture of uscfDNA derived from normal and cancerous cells.

In some embodiments, blood, plasma and serum DNA from a subject with a disease or disorder (e.g., an auto-immune disease or disorder) contains activated or inactivated genes due to differences in methylation, that can be identified using the methods of the invention.

Identification of biomarkers associated with diseases and disorders that can be determined in the circulating uscfDNA in patients is a potential diagnostic and prognostic tool. In one embodiment, methods described herein are used to determine novel biomarkers, mutations or CNVs for diseases or disorders.

### Data Processing

After isolating uscfDNA as described herein, the uscfDNA may be detected and/or analyzed by any suitable method and any suitable detection device. One or more target nucleic acids in the uscfDNA may be detected and/or analyzed. In some embodiments, the uscfDNA may potentially contain somatic mutations or novel mutations useful for identifying cancer. In some embodiments, the uscfDNA may contain methylated markers that can be used to identify auto-immunity diseases. In some embodiments, the uscfDNA may also be useful for as a global biomarker in which its increase concentration may be diagnostic of aberrations in the patient's condition. Therefore, in some embodiments, the invention includes methods of diagnosing subjects based on the identification of a biomarker in uscfDNA isolated according to the uscfDNA isolation methods of the invention.

In some embodiments, a diagnosis or the presence or absence of an outcome can be determined from the detection and/or analysis results. In some embodiments, the term “outcome” as used herein can refer to the presence, absence or total amount of one or more uscfDNA nucleic acids in the sample. In some embodiments, the term “outcome” as used herein can refer to the presence, absence or amount of a biomarker in a population of uscfDNA nucleic acids in the sample. In some embodiments, the term “outcome” as used herein can refer to an increase or decrease in the proportion of total uscfDNA nucleic acids in the sample. In some embodiments, the term “outcome” as used herein can refer to identification of a disease, disorder or condition associated with the presence, absence, biomarker or total amount of one or more uscfDNA nucleic acids in the sample. Non-limiting examples of outcomes include presence or absence of a fetus (e.g., a pregnancy test), prenatal or neonatal disorder, chromosome abnormality, chromosome aneuploidy (e.g., trisomy 21, trisomy 18, trisomy 13), a cellular proliferation condition (e.g., cancer), a cellular instability condition, an autoimmune disease or disorder and the like.

As described herein, algorithms, software, processors and/or machines, for example, can be utilized to (i) process detection data pertaining to uscfDNA nucleic acid, and/or (ii) identify the presence or absence of an outcome.

The presence or absence of an outcome may be determined for all samples tested, or in some embodiments, the presence or absence of an outcome is determined in a subset of the samples (e.g., samples from individual subjects). An outcome may be determined for about 60, 65, 70, 75, 76, 77, 78, 79, 80, 81, 82, 83, 84, 85, 86, 87, 88, 89, 90, 91, 92, 93, 94, 95, 96, 97, 98, 99%, or greater than 99%, of samples analyzed in a set. A set of samples can include any suitable number of samples, and in some embodiments, a set has about 10, 15, 20, 25, 30, 35, 40, 45, 50, 55, 60, 65, 70, 75, 80, 90, 100, 200, 300, 400, 500, 600, 700, 800, 900 or 1000 samples, or more than 1000 samples. The set may be considered with respect to samples tested in a particular period of time, and/or at a particular location. The set may be otherwise defined by, for example, age and/or ethnicity. The set may be comprised of a sample which is subdivided into subsamples or replicates all or some of which may be tested. The set may comprise a sample from the same subject collected at two different times. An outcome may be determined about 60% or more of the time for a given sample analyzed (e.g., about 65, 70, 75, 76, 77, 78, 79, 80, 81, 82, 83, 84, 85, 86, 87, 88, 89, 90, 91, 92, 93, 94, 95, 96, 97, 98, 99%, or more than 99% of the time for a given sample). Analyzing a higher number of characteristics (e.g., sequence variations) that discriminate alleles can increase the percentage of outcomes determined for the samples (e.g., discriminated in a multiplex analysis). One or more fluid samples (e.g., one or more blood samples) may be provided by a subject. One or more uscfDNA enriched samples, or two or more replicate uscfDNA enriched samples, may be isolated from a single fluid sample, and analyzed by methods described herein.

Presence or absence of an outcome can be expressed in any suitable form, and in conjunction with any suitable variable, collectively including, without limitation, ratio, deviation in ratio, frequency, distribution, probability (e.g., odds ratio, p-value), likelihood, percentage, value over a threshold, or risk factor, associated with the presence of a outcome for a subject or sample. An outcome may be provided with one or more variables, including, but not limited to, sensitivity, specificity, standard deviation, probability, ratio, coefficient of variation (CV), threshold, score, probability, confidence level, or combination of the foregoing, in certain embodiments.

One or more of ratio, sensitivity, specificity and/or confidence level may be expressed as a percentage. The percentage, independently for each variable, may be greater than about 90% (e.g., about 90, 91, 92, 93, 94, 95, 96, 97, 98 or 99%, or greater than 99% (e.g., about 99.5%, or greater, about 99.9% or greater, about 99.95% or greater, about 99.99% or greater)). Coefficient of variation (CV) in some embodiments is expressed as a percentage, and sometimes the percentage is about 10% or less (e.g., about 10, 9, 8, 7, 6, 5, 4, 3, 2 or 1%, or less than 1% (e.g., about 0.5% or less, about 0.1% or less, about 0.05% or less, about 0.01% or less)). A probability (e.g., that a particular outcome determined by an algorithm is not due to chance) in certain embodiments is expressed as a p-value, and sometimes the p-value is about 0.05 or less (e.g., about 0.05, 0.04, 0.03, 0.02 or 0.01, or less than 0.01 (e.g., about 0.001 or less, about 0.0001 or less, about 0.00001 or less, about 0.000001 or less)).

For example, scoring or a score may refer to calculating the probability that a particular outcome is actually present or absent in a subject/sample. The value of a score may be used to determine for example the variation, difference, or ratio of amplified nucleic detectable product that may correspond to the actual outcome. For example, calculating a positive score from detectable products can lead to an identification of an outcome, which is particularly relevant to analysis of single samples.

Simulated (or simulation) data can aid data processing for example by training an algorithm or testing an algorithm. Simulated data may for instance involve hypothetical various samples of different concentrations of uscfDNA in serum, plasma, saliva and the like. Simulated data may be based on what might be expected from a real population or may be skewed to test an algorithm and/or to assign a correct classification based on a simulated data set. Simulated data also is referred to herein as “virtual” data. Simulations can be performed in most instances by a computer program. One possible step in using a simulated data set is to evaluate the confidence of the identified results, i.e. how well the selected positives/negatives match the sample and whether there are additional variations. A common approach is to calculate the probability value (p-value) which estimates the probability of a random sample having better score than the selected one. As p-value calculations can be prohibitive in certain circumstances, an empirical model may be assessed, in which it is assumed that at least one sample matches a reference sample (with or without resolved variations). Alternatively other distributions such as Poisson distribution can be used to describe the probability distribution.

An algorithm can assign a confidence value to the true positives, true negatives, false positives and false negatives calculated. The assignment of a likelihood of the occurrence of a outcome can also be based on a certain probability model.

Simulated data often is generated in an in silico process. As used herein, the term “in silico” refers to research and experiments performed using a computer. In silico methods include, but are not limited to, molecular modeling studies, karyotyping, genetic calculations, biomolecular docking experiments, and virtual representations of molecular structures and/or processes, such as molecular interactions.

As used herein, a “data processing routine” refers to a process that can be embodied in software that determines the biological significance of acquired data (i.e., the ultimate results of an assay). For example, a data processing routine can determine the amount of each nucleotide sequence species based upon the data collected. A data processing routine also may control an instrument and/or a data collection routine based upon results determined. A data processing routine and a data collection routine often are integrated and provide feedback to operate data acquisition by the instrument, and hence provide assay-based judging methods provided herein.

As used herein, software refers to computer readable program instructions that, when executed by a computer, perform computer operations. Typically, software is provided on a program product containing program instructions recorded on a computer readable medium, including, but not limited to, magnetic media including floppy disks, hard disks, and magnetic tape; and optical media including CD-ROM discs, DVD discs, magneto-optical discs, and other such media on which the program instructions can be recorded.

Different methods of predicting abnormality or normality can produce different types of results. For any given prediction, there are four possible types of outcomes: true positive, true negative, false positive or false negative. The term “true positive” as used herein refers to a subject correctly diagnosed as having a outcome. The term “false positive” as used herein refers to a subject wrongly identified as having a outcome. The term “true negative” as used herein refers to a subject correctly identified as not having a outcome. The term “false negative” as used herein refers to a subject wrongly identified as not having a outcome. Two measures of performance for any given method can be calculated based on the ratios of these occurrences: (i) a sensitivity value, the fraction of predicted positives that are correctly identified as being positives (e.g., the fraction of nucleotide sequence sets correctly identified by level comparison detection/determination as indicative of outcome, relative to all nucleotide sequence sets identified as such, correctly or incorrectly), thereby reflecting the accuracy of the results in detecting the outcome; and (ii) a specificity value, the fraction of predicted negatives correctly identified as being negative (the fraction of nucleotide sequence sets correctly identified by level comparison detection/determination as indicative of chromosomal normality, relative to all nucleotide sequence sets identified as such, correctly or incorrectly), thereby reflecting accuracy of the results in detecting the outcome.

The term “sensitivity” as used herein refers to the number of true positives divided by the number of true positives plus the number of false negatives, where sensitivity (sens) may be within the range of 0≤sens≤1. Ideally, method embodiments herein have the number of false negatives equaling zero or close to equaling zero, so that no subject is wrongly identified as not having at least one outcome when they indeed have at least one outcome. Conversely, an assessment often is made of the ability of a prediction algorithm to classify negatives correctly, a complementary measurement to sensitivity. The term “specificity” as used herein refers to the number of true negatives divided by the number of true negatives plus the number of false positives, where sensitivity (spec) may be within the range of 0≤spec≤1. Ideally, methods embodiments herein have the number of false positives equaling zero or close to equaling zero, so that no subject wrongly identified as having at least one outcome when they do not have the outcome being assessed. Hence, a method that has sensitivity and specificity equaling one, or 100%, sometimes is selected.

One or more prediction algorithms may be used to determine significance or give meaning to the detection data collected under variable conditions that may be weighed independently of or dependently on each other. The term “variable” as used herein refers to a factor, quantity, or function of an algorithm that has a value or set of values. For example, a variable may be the design of a set of amplified nucleic acid species, the number of sets of amplified nucleic acid species, type of outcome assayed, and the like.

Any suitable type of method or prediction algorithm may be utilized to give significance to the data of the present technology within an acceptable sensitivity and/or specificity. For example, prediction algorithms such as Mann-Whitney U Test, binomial test, log odds ratio, Chi-squared test, z-test, t-test, ANOVA (analysis of variance), regression analysis, neural nets, fuzzy logic, Hidden Markov Models, multiple model state estimation, and the like may be used. One or more methods or prediction algorithms may be determined to give significance to the data having different independent and/or dependent variables of the present technology. And one or more methods or prediction algorithms may be determined not to give significance to the data having different independent and/or dependent variables of the present technology. One may design or change parameters of the different variables of methods described herein based on results of one or more prediction algorithms (e.g., number of sets analyzed, types of nucleotide species in each set).

Several algorithms may be chosen to be tested. These algorithms then can be trained with raw data. For each new raw data sample, the trained algorithms will assign a classification to that sample (e.g., trisomy or normal). Based on the classifications of the new raw data samples, the trained algorithms' performance may be assessed based on sensitivity and specificity. Finally, an algorithm with the highest sensitivity and/or specificity or combination thereof may be identified.

Provided are methods for identifying the presence or absence of an outcome that comprise: (a) providing a system, wherein the system comprises distinct software modules, and wherein the distinct software modules comprise a signal detection module, a logic processing module, and a data display organization module; (b) detecting signal information indicating the presence, absence or amount of enriched nucleic acid; (c) receiving, by the logic processing module, the signal information; (d) calling the presence or absence of an outcome by the logic processing module; and (e) organizing, by the data display organization model in response to being called by the logic processing module, a data display indicating the presence or absence of the outcome.

Provided also are methods for identifying the presence or absence of an outcome, which comprise providing signal information indicating the presence, absence or amount of enriched nucleic acid; providing a system, wherein the system comprises distinct software modules, and wherein the distinct software modules comprise a signal detection module, a logic processing module, and a data display organization module; receiving, by the logic processing module, the signal information; calling the presence or absence of an outcome by the logic processing module; and, organizing, by the data display organization model in response to being called by the logic processing module, a data display indicating the presence or absence of the outcome.

Provided also are methods for identifying the presence or absence of an outcome, which comprise providing a system, wherein the system comprises distinct software modules, and wherein the distinct software modules comprise a signal detection module, a logic processing module, and a data display organization module; receiving, by the logic processing module, signal information indicating the presence, absence or amount of enriched nucleic acid; calling the presence or absence of an outcome by the logic processing module; and, organizing, by the data display organization model in response to being called by the logic processing module, a data display indicating the presence or absence of the outcome.

By “providing signal information” is meant any manner of providing the information, including, for example, computer communication means from a local, or remote site, human data entry, or any other method of transmitting signal information. The signal information may be generated in one location and provided to another location.

By “obtaining” or “receiving” signal information is meant receiving the signal information by computer communication means from a local, or remote site, human data entry, or any other method of receiving signal information. The signal information may be generated in the same location at which it is received, or it may be generated in a different location and transmitted to the receiving location.

By “indicating” or “representing” the amount is meant that the signal information is related to, or correlates with, for example, the amount of enriched nucleic acid or presence or absence of enriched nucleic acid. The information may be, for example, the calculated data associated with the presence or absence of enriched nucleic acid as obtained, for example, after converting raw data obtained by mass spectrometry.

Also provided are computer program products, such as, for example, a computer program products comprising a computer usable medium having a computer readable program code embodied therein, the computer readable program code adapted to be executed to implement a method for identifying the presence or absence of an outcome, which comprises (a) providing a system, wherein the system comprises distinct software modules, and wherein the distinct software modules comprise a signal detection module, a logic processing module, and a data display organization module; (b) detecting signal information indicating the presence, absence or amount of enriched nucleic acid; (c) receiving, by the logic processing module, the signal information; (d) calling the presence or absence of an outcome by the logic processing module; and, organizing, by the data display organization model in response to being called by the logic processing module, a data display indicating the presence or absence of the outcome.

Also provided are computer program products, such as, for example, computer program products comprising a computer usable medium having a computer readable program code embodied therein, the computer readable program code adapted to be executed to implement a method for identifying the presence or absence of an outcome, which comprises providing a system, wherein the system comprises distinct software modules, and wherein the distinct software modules comprise a signal detection module, a logic processing module, and a data display organization module; receiving signal information indicating the presence, absence or amount of enriched nucleic acid; calling the presence or absence of an outcome by the logic processing module; and, organizing, by the data display organization model in response to being called by the logic processing module, a data display indicating the presence or absence of the outcome.

Signal information may be, for example, mass spectrometry data obtained from mass spectrometry of uscfDNA, or of a uscfDNA enriched sample. As the uscfDNA may be amplified into a nucleic acid that is detected, the signal information may be detection information, such as mass spectrometry data, obtained from uscf nucleic acid or stoichiometrically amplified nucleic acid from the uscf nucleic acid, for example. The mass spectrometry data may be raw data, such as, for example, a set of numbers, or, for example, a two dimensional display of the mass spectrum. The signal information may be converted or transformed to any form of data that may be provided to, or received by, a computer system. The signal information may also, for example, be converted, or transformed to identification data or information representing an outcome. An outcome may be, for example, a fetal allelic ratio, or a particular chromosome number in fetal cells. Where the chromosome number is greater or less than in euploid cells, or where, for example, the chromosome number for one or more of the chromosomes, for example, 21, 18, or 13, is greater than the number of other chromosomes, the presence of a chromosomal disorder may be identified.

Also provided is a machine for identifying the presence or absence of an outcome wherein the machine comprises a computer system having distinct software modules, and wherein the distinct software modules comprise a signal detection module, a logic processing module, and a data display organization module, wherein the software modules are adapted to be executed to implement a method for identifying the presence or absence of an outcome, which comprises (a) detecting signal information indicating the presence, absence or amount of uscf nucleic acid; (b) receiving, by the logic processing module, the signal information; (c) calling the presence or absence of an outcome by the logic processing module, wherein a ratio of alleles different than a normal ratio is indicative of a chromosomal disorder; and (d) organizing, by the data display organization model in response to being called by the logic processing module, a data display indicating the presence or absence of the outcome. The machine may further comprise a memory module for storing signal information or data indicating the presence or absence of a chromosomal disorder. Also provided are methods for identifying the presence or absence of an outcome, wherein the methods comprise the use of a machine for identifying the presence or absence of an outcome.

Also provided are methods identifying the presence or absence of an outcome that comprises: (a) detecting signal information, wherein the signal information indicates presence, absence or amount of uscf nucleic acid; (b) transforming the signal information into identification data, wherein the identification data represents the presence or absence of the outcome, whereby the presence or absence of the outcome is identified based on the signal information; and (c) displaying the identification data.

Also provided are methods for identifying the presence or absence of an outcome that comprises:
(a) providing signal information indicating the presence, absence or amount of uscfDNA; (b) transforming the signal information representing into identification data, wherein the identification data represents the presence or absence of the outcome, whereby the presence or absence of the outcome is identified based on the signal information; and (c) displaying the identification data.

Also provided are methods for identifying the presence or absence of an outcome that comprises:
(a) receiving signal information indicating the presence, absence or amount of uscfDNA; (b) transforming the signal information into identification data, wherein the identification data represents the presence or absence of the outcome, whereby the presence or absence of the outcome is identified based on the signal information; and (c) displaying the identification data.

For purposes of these, and similar embodiments, the term “signal information” indicates information readable by any electronic media, including, for example, computers that represent data derived using the present methods. For example, “signal information” can represent the amount of uscf nucleic acid or amplified nucleic acid. Signal information, such as in these examples, that represents physical substances may be transformed into identification data, such as a visual display that represents other physical substances, such as, for example, a chromosome disorder, or a chromosome number. Identification data may be displayed in any appropriate manner, including, but not limited to, in a computer visual display, by encoding the identification data into computer readable media that may, for example, be transferred to another electronic device (e.g., electronic record), or by creating a hard copy of the display, such as a print out or physical record of information. The information may also be displayed by auditory signal or any other means of information communication. In some embodiments, the signal information may be detection data obtained using methods to detect uscf nucleic acid.

Once the signal information is detected, it may be forwarded to the logic-processing module. The logic-processing module may “call” or “identify” the presence or absence of an outcome.

Provided also are methods for transmitting genetic information to a subject, which comprise identifying the presence or absence of an outcome wherein the presence or absence of the outcome has been determined from determining the presence, absence or amount of uscf nucleic acid from a sample from the subject; and transmitting the presence or absence of the outcome to the subject. A method may include transmitting prenatal genetic information to a human pregnant female subject, and the outcome may be presence or absence of a chromosome abnormality or aneuploidy, in certain embodiments.

The term “identifying the presence or absence of an outcome” or “an increased risk of an outcome,” as used herein refers to any method for obtaining such information, including, without limitation, obtaining the information from a laboratory file. A laboratory file can be generated by a laboratory that carried out an assay to determine the presence or absence of an outcome. The laboratory may be in the same location or different location (e.g., in another country) as the personnel identifying the presence or absence of the outcome from the laboratory file. For example, the laboratory file can be generated in one location and transmitted to another location in which the information therein will be transmitted to the subject. The laboratory file may be in tangible form or electronic form (e.g., computer readable form), in certain embodiments.

The term “transmitting the presence or absence of the outcome to the subject” or any other information transmitted as used herein refers to communicating the information to the subject, or family member, guardian or designee thereof, in a suitable medium, including, without limitation, in verbal, document, or file form.

Also provided are methods for providing to a subject a medical prescription based on genetic information, which comprise identifying the presence or absence of an outcome, wherein the presence or absence of the outcome has been determined from the presence, absence or amount of uscf nucleic acid from a sample from the subject; and providing a medical prescription based on the presence or absence of the outcome to the subject.

The term “providing a medical prescription based on genetic information” refers to communicating the prescription to the subject, or family member, guardian or designee thereof, in a suitable medium, including, without limitation, in verbal, document or file form. The medical prescription may be for any course of action determined by, for example, a medical professional upon reviewing the uscfDNA genetic information. For example, the medical prescription may be for the subject to undergo additional testing or confirmatory testing. In yet another example, the medical prescription may be medical advice to not undergo further testing.

Also provided are files, such as, for example, a file comprising the presence or absence of outcome for a subject, wherein the presence or absence of the outcome has been determined from the presence, absence or amount of uscf nucleic acid in a sample from the subject. The file may be, for example, but not limited to, a computer readable file, a paper file, or a medical record file.

Computer program products include, for example, any electronic storage medium that may be used to provide instructions to a computer, such as, for example, a removable storage device, CD-ROMS, a hard disk installed in hard disk drive, signals, magnetic tape, DVDs, optical disks, flash drives, RAM or floppy disk, and the like.

The systems discussed herein may further comprise general components of computer systems, such as, for example, network servers, laptop systems, desktop systems, handheld systems, personal digital assistants, computing kiosks, and the like. The computer system may comprise one or more input means such as a keyboard, touch screen, mouse, voice recognition or other means to allow the user to enter data into the system. The system may further comprise one or more output means such as a CRT or LCD display screen, speaker, FAX machine, impact printer, inkjet printer, black and white or color laser printer or other means of providing visual, auditory or hardcopy output of information.

The input and output means may be connected to a central processing unit which may comprise among other components, a microprocessor for executing program instructions and memory for storing program code and data. In some embodiments the methods may be implemented as a single user system located in a single geographical site. In other embodiments methods may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by the provider or it may be implemented as an Internet based service where the user accesses a web page to enter and retrieve information.

The various software modules associated with the implementation of the present products and methods can be suitably loaded into the computer system as desired, or the software code can be stored on a computer-readable medium such as a floppy disk, magnetic tape, or an optical disk, or the like. In an online implementation, a server and web site maintained by an organization can be configured to provide software downloads to remote users. As used herein, “module,” including grammatical variations thereof, means, a self-contained functional unit which is used with a larger system. For example, a software module is a part of a program that performs a particular task. Thus, provided herein is a machine comprising one or more software modules described herein, where the machine can be, but is not limited to, a computer (e.g., server) having a storage device such as floppy disk, magnetic tape, optical disk, random access memory and/or hard disk drive, for example.

The present methods may be implemented using hardware, software or a combination thereof and may be implemented in a computer system or other processing system. An example computer system may include one or more processors. A processor can be connected to a communication bus. The computer system may include a main memory, sometimes random access memory (RAM), and can also include a secondary memory. The secondary memory can include, for example, a hard disk drive and/or a removable storage drive, representing a floppy disk drive, a magnetic tape drive, an optical disk drive, memory card etc. The removable storage drive reads from and/or writes to a removable storage unit in a well-known manner. A removable storage unit includes, but is not limited to, a floppy disk, magnetic tape, optical disk, etc. which is read by and written to by, for example, a removable storage drive. As will be appreciated, the removable storage unit includes a computer usable storage medium having stored therein computer software and/or data.

Alternatively, secondary memory may include other similar means for allowing computer programs or other instructions to be loaded into a computer system. Such means can include, for example, a removable storage unit and an interface device. Examples of such can include a program cartridge and cartridge interface (such as that found in video game devices), a removable memory chip (such as an EPROM, or PROM) and associated socket, and other removable storage units and interfaces which allow software and data to be transferred from the removable storage unit to a computer system.

The computer system may also include a communications interface. A communications interface allows software and data to be transferred between the computer system and external devices. Examples of communications interface can include a modem, a network interface (such as an Ethernet card), a communications port, a PCMCIA slot and card, etc. Software and data transferred via communications interface are in the form of signals, which can be electronic, electromagnetic, optical or other signals capable of being received by communications interface. These signals are provided to communications interface via a channel. This channel carries signals and can be implemented using wire or cable, fiber optics, a phone line, a cellular phone link, an RF link and other communications channels. Thus, in one example, a communications interface may be used to receive signal information to be detected by the signal detection module.

In a related aspect, the signal information may be input by a variety of means, including but not limited to, manual input devices or direct data entry devices (DDEs). For example, manual devices may include, keyboards, concept keyboards, touch sensitive screens, light pens, mouse, tracker balls, joysticks, graphic tablets, scanners, digital cameras, video digitizers and voice recognition devices. DDEs may include, for example, bar code readers, magnetic strip codes, smart cards, magnetic ink character recognition, optical character recognition, optical mark recognition, and turnaround documents. In one embodiment, an output from a gene or chip reader my serve as an input signal.

EFIRM Based Analysis of uscfDNA

In some embodiments, uscfDNA isolated according to the method of the invention can be applied to an EFIRM system for the detection of biomarkers. In some embodiments, the EFIRM assay includes a multiplexing electrochemical sensor for detecting biomarkers. The device utilizes a small sample volume with high accuracy. In addition, multiple markers can be measured simultaneously on the device with single sample loading. The device may significantly reduce the cost to the health care system, by decreasing the burden of patients returning to clinics and laboratories.

In one embodiment, the electrochemical sensor is an array of electrode chips (EZ Life Bio, USA). In one embodiment, each unit of the array has a working electrode, a counter electrode, and a reference electrode. The three electrodes may be constructed of bare gold or other conductive material before the reaction, such that the specimens may be immobilized on the working electrode. Electrochemical current can be measured between the working electrode and counter electrode under the potential between the working electrode and the reference electrode. The potential profile can be a constant value, a linear sweep, or a cyclic square wave, for example. An array of plastic wells may be used to separate each three-electrode set, which helps avoid the cross contamination between different sensors. In one embodiment, a three-electrode set is in each well of a 96 well gold electrode plate. A conducting polymer may also be deposited on the working electrodes as a supporting film, and in some embodiments, as a surface to functionalize the working electrode. As contemplated herein, any conductive polymer may be used, such as polypyrroles, polanilines, polyacetylenes, polyphenylenevinylenes, polythiophenes and the like.

In one embodiment, a cyclic square wave electric field is generated across the electrode within the sample well. In certain embodiments, the square wave electric field is generated to aid in polymerization of one or more capture probes to the polymer of the sensor.

In certain embodiments, the square wave electric field is generated to aid in the hybridization of the capture probes with the marker and/or detector probe. The positive potential in the csw E-field helps the molecules accumulate onto the working electrode, while the negative potential removes the weak nonspecific binding, to generate enhanced specificity. Further, the flapping between positive and negative potential across the cyclic square wave also provides superior mixing during incubation, without disruption of the desired specific binding, which accelerates the binding process and results in a faster test or assay time. In one embodiment, a square wave cycle may consist of a longer low voltage period and a shorter high voltage period, to enhance binding partner hybridization within the sample. While there is no limitation to the actual time periods selected, examples include 0.15 to 60 second low voltage periods and 0.1 to 60 second high voltage periods. In one embodiment, each square-wave cycle consists of 1 s at low voltage and 1 s at high voltage. For hybridization, the low voltage may be around-200 mV and the high voltage may be around +500 mV. In some embodiments, the total number of square wave cycles may be between 2-50. In one embodiment, 5 cyclic square-waves are applied for each surface reaction. With the csw E-field, both the polymerization and hybridization are finished on the same chip within minutes. In some embodiments, the total detection time from sample loading is less than 30 minutes. In other embodiments, the total detection time from sample loading is less than 20 minutes. In other embodiments, the total detection time from sample loading is less than 10 minutes. In other embodiments, the total detection time from sample loading is less than 5 minutes. In other embodiments, the total detection time from sample loading is less than 2 minutes. In other embodiments, the total detection time from sample loading is less than 1 minute.

A multi-channel electrochemical reader (EZ Life Bio) controls the electrical field applied onto the array sensors and reports the amperometric current simultaneously. In practice, solutions can be loaded onto the entire area of the three-electrode region including the working, counter, and reference electrodes, which are confined and separated by the array of plastic wells. After each step, the electrochemical sensors can be rinsed with ultrapure water or other washing solution and then dried, such as under pure N₂. In some embodiments, the sensors are single use, disposable sensors. In other embodiment, the sensors are reusable.

### Determining Effectiveness of Therapy or Prognosis

In one aspect, the level of one or more uscfDNA, or a biomarker identified therein, in a biological sample of a patient is used to monitor the effectiveness of treatment or the prognosis of disease. In some embodiments, the level of one or more uscfDNA, or a biomarker identified therein, in a test sample obtained from a treated patient can be compared to the level from a reference sample obtained from that patient before initiation of a treatment. Clinical monitoring of treatment typically entails that each patient serves as his or her own baseline control. In some embodiments, test samples are obtained at multiple time points following administration of the treatment. In these embodiments, measurement of the level of one or more uscfDNA, or a biomarker identified therein, in the test samples provides an indication of the extent and duration of in vivo effect of the treatment.

Measurement of the level of one or more uscfDNA, may allow for the course of treatment of a disease to be monitored. The effectiveness of a treatment regimen for a disease can be monitored by detecting one or more uscfDNA in an effective amount from samples obtained from a subject over time and comparing the detected level of one or more uscfDNA. For example, a first sample can be obtained before the subject receives treatment and one or more subsequent samples are taken after or during treatment of the subject. Changes in uscfDNA levels across the samples may provide an indication as to the effectiveness of the therapy.

In some embodiments, the disclosure provides a method for monitoring the levels of uscfDNA in response to treatment. For example, in certain embodiments, the disclosure provides for a method of determining the efficacy of treatment in a subject, by measuring the levels of one or more uscfDNA as described herein. In some embodiments, the level of the one or more uscfDNA can be measured over time, where the level at one timepoint after the initiation of treatment is compared to the level at another timepoint after the initiation of treatment. In some embodiments, the level of the one or more uscfDNA can be measured over time, where the level at one timepoint after the initiation of treatment is compared to the level before initiation of treatment.

In some embodiments, uscfDNA levels can be used to identify therapeutics or drugs that are appropriate for a specific subject. For example, a test sample from the subject can be exposed to a therapeutic agent or a drug, and the level of one or more uscfDNA can be determined. UscfDNA levels can be compared to a sample derived from the subject before and after treatment or exposure to a therapeutic agent or a drug or can be compared to samples derived from one or more subjects who have shown improvements relative to a disease as a result of such treatment or exposure. Thus, in one aspect, the disclosure provides a method of assessing the efficacy of a therapy with respect to a subject comprising taking a first measurement of uscfDNA or a uscfDNA panel in a first sample from the subject; effecting the therapy with respect to the subject; taking a second measurement of the uscfDNA or uscfDNA panel in a second sample from the subject and comparing the first and second measurements to assess the efficacy of the therapy.

Accordingly, treatments or therapeutic regimens for use in can be selected based on the amounts of a specific uscfDNA or a uscfDNA panel in samples obtained from the subjects and compared to a reference value. Two or more treatments or therapeutic regimens can be evaluated in parallel to determine which treatment or therapeutic regimen would be the most efficacious for use in a subject to delay onset, or slow progression of a disease. In various embodiments, a recommendation is made on whether to initiate or continue treatment of a disease.

A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example, prognosis can be expressed as a percent chance that a patient will survive after one year, five years, ten years or the like. Alternatively, prognosis may be expressed as the number of years, on average that a patient can expect to survive as a result of a condition or disease. The prognosis of a patient may be considered as an expression of relativism, with many factors affecting the ultimate outcome. For example, for patients with certain conditions, prognosis can be appropriately expressed as the likelihood that a condition may be treatable or curable, or the likelihood that a disease will go into remission, whereas for patients with more severe conditions, prognosis may be more appropriately expressed as likelihood of survival for a specified period of time. Additionally, a change in a clinical factor from a baseline level may impact a patient's prognosis, and the degree of change in level of the clinical factor may be related to the severity of adverse events. Statistical significance is often determined by comparing two or more populations and determining a confidence interval and/or a p value.

Multiple determinations of uscfDNA levels can be made, and a temporal change in uscfDNA level can be used to determine a prognosis. For example, comparative measurements are made of the uscfDNA level in a patient at multiple time points, and a comparison of the uscfDNA level at two or more time points may be indicative of a particular prognosis.

In certain embodiments, other prognostic factors may be combined with the uscfDNA level or other biomarkers in the algorithm to determine prognosis with greater accuracy. Exemplary additional prognostic factors may include one or more prognostic factors selected from the group consisting of cytogenetics, performance status, age, gender and contemporary diagnosis.

### Treatments

In one aspect, the disclosure provides a method of diagnosing, treating or preventing a disease or disorder associated with a biomarker identified from analysis of uscfDNA, an altered level of a specific uscfDNA or a general increase or decrease of total uscfDNA. In some embodiments, the method comprises administering to the subject an effective amount of a pharmaceutical agent for the treatment of a disease or disorder identified associated with a biomarker identified from analysis of uscfDNA, an altered level of a specific uscfDNA or a general increase or decrease of total uscfDNA.

### Kits

The present invention further includes an assay kit containing the components for performing a uscfDNA isolation assay of the invention, including, but not limited to, reagents, enzymes, buffers, separation beads, tubes, and instructions for the set-up, performance, monitoring, and interpretation of the assays of the present invention. Optionally, the kit may include control reagents and reagents for the detection of at least one biomarkers.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Plasma Contains Ultrashort Single-Stranded DNA in Addition to Nucleosomal Cell-Free DNA

Plasma cell-free DNA is being widely explored as a biomarker for clinical screening. Currently, methods are optimized for the extraction and detection of double-stranded mono-nucleosomal cell-free DNA of ˜160 bp in length. BRcfDNA-Seq, a single-stranded cell-free DNA next-generation sequencing pipeline, was developed which bypasses previous limitations to reveal a population of ultrashort single-stranded cell-free DNA in human plasma. This species has a modal size of 50 nt and is distinctly separate from mono-nucleosomal cell-free DNA. Treatment with single-stranded and double-stranded specific nucleases suggest that ultrashort cell-free DNA is primarily single-stranded. It is distributed evenly across chromosomes and has a similar distribution profile over functional elements as the genome, albeit with an enrichment over promoters, exons, and introns which may be suggestive of a terminal state of genome degradation. The examination of this cfDNA species could reveal new features of cell death pathways or it can be used for cell-free DNA biomarker discovery.

The revelation that there are two distinct populations of cfDNA opens up several new avenues for scientific exploration. Firstly, the field of molecular diagnostics must now consider the uscfDNA population, in conjunction with conventional mncfDNA, for biomarker identification and diagnosis. Therefore, in liquid biopsy for cancer detection, uscfDNA could provide a new resource of available biomarkers. It has long been observed that in late-stage cancer, not only does the concentration of cell-free DNA increase, the average fragment length also decreases by 10-20 bp (Lapin et al., J Transl Med, 2018, 16). Mutation containing cell-free DNA is consistently shorter than wildtype DNA and this skewed impression fragment size in late-stage cancer is likely due to the increased ratio of cancer cells undergoing apoptosis (Mouliere et al., Sci Transl Med, 2018, 10). These previous studies, however, only utilize extraction and DNA-quantification methods that consider the double-stranded mncfDNA population. Whether this observed pattern in late-stage cancer donors is mirrored by uscfDNA is not clear. Conversely, a study on cfDNA from pancreatic patient plasma using single-stranded library preparation (extracted with the equivalent of QiaC) showed that earlier stages are actually associated with shorter fragments (Liu et al., EBioMedicine, 2019, (41) 345-356). This apparent contradiction may hint that size profiles and concentrations of these two populations of cfDNA may have contrasting trajectory during between the healthy, early-stage, and late-stage cancer phases.

Since the uscfDNA has enriched promoter, exon, and intron elements compared with the mncfDNA, uscfDNA could be a better reservoir for specific biomarker sequences. Most genetic aberrations in diseases are associated with coding regions and not the intergenic sequences enriched in mncfDNA. There may be merit in using single-stranded library preparation kits without the initial heatshock if investigators wish to enrich uscfDNA fragments in their final library. Although in theory, dsDNase treatment should enrich the library for uscfDNA, it actually lowers the percent of promoters, introns, and exons by possibly adding degraded mncfDNA molecules to the uscfDNA size pool.

When looking for rare mutations, the short footprint of uscfDNA should be considered for calculations regarding genomic coverage. Due to uscfDNA having shorter reads, libraries with substantial uscfDNA population will require more total reads to achieve the same genomic coverage as a mncfDNA dominant library (Desai et al., PLOS One, 2013, 8). Therefore, target capture to enrich the coverage in certain regions will be required for any rare mutation detection. By applying target-capture enrichment, evidence was found that ultrashort circulating tumor DNA contained in plasma from non-small cell lung carcinoma patients can also harbor mutations corresponding to the mncfDNA and tissue genotyping (Li et al., Cancers, 2020, (12) 2041). However, in contrast to the methodology presented here, the pipeline was not optimized for single-strand DNA. By incorporating this BRcfDNA-Seq methodology, how uscfDNA fragment patterns are altered in different disease states in clinically-focused studies can be actively explored.

Secondly, uscfDNA introduces new potential biological insights in cfDNA biology. Previously, the functions of RNA, a prominent single-stranded entity, are well described. RNA is involved in transcription, amino-acid transfer, protein-complexes, gene expression, and signal-transfer via exosomes. By comparison, circulating ssDNA biology has been largely unexplored, and it is plausible that ssDNA may have more functions than initially thought. In molecular biology, there is limited technology to evaluate ssDNA. With the development of BRcfDNA-Seq, future studies interested in the assessment of ultrashort single-stranded DNA molecules is possible. In this regard, there is merit in exploring how uscfDNA plays a role in normal physiology and how it may change with age in comparison to the mncfDNA population (Teo et al., Aging Cell, 2019, 18).

In regards to its origins, based on the data presented here, uscfDNA appears to be involved in the cell death pathways for the disposal of genomic DNA. Extensive literature has described the origins of mncfDNA as a byproduct of genomic DNA degradation (Burnham et al., Sci Rep, 2016, 6; Nagata et al., Cell Death Differ, 2003, (10) 108-116). Based on the data provided, the genomic coverage of uscfDNA maps evenly amongst the chromosomes in the genome mirroring the pattern of mncfDNA. However, examination of the function elements of uscfDNA provides additional insights since uscfDNA closer resembled the genomic profile but with a marked enrichment in promoter sequences at 50 nt. The observed enrichment may be suggestive of originating from transcription factor-bound complexes to one strand of DNA (Tomonaga and Levens, Proc Natl Acad Sci, 1996, (93) 5830-5835). In contrast, the mncfDNA fragments had an observed decrease in exon, intron, and promoter sequences. These coding regions would be expected to be accessible for active transcription and susceptible to initial nuclease degradation unlike the nucleosomal-protected intergenic sequences. Therefore, uscfDNA could be derived from both exposed regions of the genome and eventual metabolism of nucleosome-protected mncfDNA. Recent work has begun describing possible nucleases such as DNasel, DNASEIL3, and DFFB, that contribute the regulation of mncfDNA processing (Han et al., Am J Hum Genet, 2020, (106) 202-214). Since BRcfDNA-Seq can now readily detect and analyze uscfDNA in biological samples, it is paramount to explore the nucleases which regulate its appearance in blood.

Aside from part of a degradation pathway it is plausible that that uscfDNA could be involved in biological processes. Although not yet described in eukaryotes, the bacteria genome contain “retrons” sequences which code for a special type of reverse transcriptase and a non-coding RNA sequence to generate DNA/RNA hybrid called multicopy single-stranded DNA (msDNA) (Inouye and Inouye, Curr Opin Genet Dev, 1993, (3) 713-718; Schubert et al., Proceedings of the National Academy of Sciences, 2021, 118). The retron ssDNA thought to be part of the bacterial immune system and helps to detect for invading viruses (Millman et al., Cell, 2020, (183) 1551-1561). Some msDNA have been described to be as short as 48 nt so it is conceivable that an eukaryotic version may contribute to the uscfDNA pool in plasma where the RNA component has already degraded (Mao et al., J Bacteriol, 1997, (179) 7865-7868).

Based on the functional peak analysis it appears although QiaM and SPRI can recover uscfDNA in plasma, they may be recovering a different population profile. It appears that QiaM may be enriched for promoter and exon sequences, but size efficiency experiments indicates that SPRI has greater recovery of 30-50 nt uscfDNA. However, sequences shorter than 50 bp may have greater intergenic proportion which would result in the dilution of sequences in coding regions for SPRI extracted samples.

In conclusion, the data presented herein demonstrate the BRcfDNA-Seq pipeline reveals the presence of a unique class of ultrashort single-stranded cell-free DNA of nuclear origin with a modal size of 50 nt. Careful examination of uscfDNA may likely provide new opportunities in molecular diagnostics and cfDNA biology in the future.

The Materials and Methods used for the Experiments are now described

### Clinical Samples.

Plasma from healthy donors was commercially purchased from Innovative Research (IPLASK2E10ML). One donor provided whole blood collected into three vacutainers, K2EDTA, StreckDNA, and StreckRNA (Streck, 218961 and 230460). According to vendor instructions, whole blood was spun at 5000×G for 15 minutes and plasma was removed using a plasma extractor. Age and gender of the donors can be found in Table 1.

TABLE 1
Plasma Donor Information

| Assay | Gender | Age |
|---|---|---|
| Digestions Donor 1 | Male | 47 |
| Digestions Donor 2 | Female | 57 |
| Digestions Donor 3 | Male | 35 |
| Healthy 10 Replicate Donor 1 | Male | 45 |
| Healthy 10 Replicate Donor 2 | Male | 18 |
| Healthy 10 Replicate Donor 3 | Male | 23 |
| Healthy 10 Replicate Donor 4 | Male | 26 |
| Healthy 10 Replicate Donor 5 | Male | 38 |
| Healthy 10 Replicate Donor 6 | Male | 33 |
| Healthy 10 Replicate Donor 7 | Male | 22 |
| Healthy 10 Replicate Donor 8 | Male | 37 |
| Healthy 10 Replicate Donor 9 | Male | 27 |
| Healthy 10 Replicate Donor 10 | Male | 41 |
| Healthy Donor for QiaM on QiaC Flowthrough 1 | Male | 19 |
| Healthy Donor for QiaM on QiaC Flowthrough 2 | Male | 25 |

### Nucleic Acid Extraction.

1 mL of plasma was extracted with three different methods. Using the QIAmp Circulating Nucleic Acid Kit (Qiagen, 55114) we followed two of the manufacturer protocol: Purification of Circulating Nucleic Acids from 1 mL of Plasma (QiaC) and Purification of Circulating microRNA from 1 ml of Plasma (QiaM). Proteinase-K digestion was carried out as instructed. Carrier RNA was not used. The ATL Lysis buffer (Qiagen, 19076) was used as indicated in the microRNA protocol. The final elution volume was 40 μl.

In the magnetic bead-based uscfDNA extraction, 100 μL of Proteinase K (20 mg/mL, Zymogen, D3001-2-1215) and 56 μL 20% SDS (Invitrogen, AM9820) was added to 1 mL of human plasma and incubated for 30 minutes at 60° C. After cooling to ambient room temperature, 540 μL SPRI-select beads (Beckman Coulter, B22318) and 3000 μL of 100% isopropanol (Fisher, BP26181) were added to the plasma and incubated for 10 minutes on the benchtop. The plasma was then centrifuged at 4000×G for five minutes. The supernatant was removed and discarded. The pellet was resuspended using 1 mL of 1×TE Buffer (Invitrogen, AM9848) and divided into 500 μl aliquots into two phase lock tubes (Quantabio, 10847-802). An equal volume (500 μL) of phenol:chloroform:isoamyl alcohol with equilibrium buffer was added (Sigma, P2069-100 mL) and contents were vortexed for 15 seconds. The tubes were then centrifuged at 19000×G for five minutes. This was repeated twice (vortexed and centrifuged). The upper clear supernatant was pipetted and transferred to a 15 ml conical tube SPRI-select beads and 3000 μL of 100% isopropanol were added to the plasma and incubated for 10 minutes on the benchtop. The tube was placed on a magnetic rack for five minutes to allow for the beads to migrate. The supernatant was discarded and the beads were washed twice with 5 ml of 85% ethanol. Once the second ethanol wash was removed the beads were left to air dry for 10 minutes. The beads were then resuspended in 30 μL of elution buffer (Qiagen, 19086) and incubated for 2 minutes. After the beads were transferred to a 1.5 mL tube and magnet rack to separate the beads. Once the solution was clear (˜2 minutes) the 30 μL of elution was transferred to another 1.5 mL tube and combined with 1 μL of 20 mg/ml glycogen (Thermo, R0561), 44 μL of 1×TE Buffer, 25 μL of 3M sodium acetate (Quality Biological INC, 50-751-7660), 250 μL of 100% ethanol and placed at −80° C. overnight. The tube was then centrifuged at 19000×G for 15 minutes. The supernatant was removed and replaced with 200 μL of 80% ethanol. This was done 2 more times. The supernatant was removed and the pellet was resuspended in a 30 μL of elution buffer and combined with 90 μL of SPRI-select beads, 90 μL of 100% isopropanol and incubated for 10 minutes. The tube was placed on a magnetic rack for five minutes to allow for the beads to migrate. The supernatant was discarded and the beads were washed twice with 200 μL of 80% ethanol. Once the second ethanol wash was removed the beads were left to air dry for 10 minutes. The beads were then resuspended in 40 μL of Qiagen elution buffer.

### Library Preparations.

Single-stranded DNA library preparation was performed using the SRSLY™ PicoPlus DNA NGS Library Preparation Base Kit with the SRSLY 12 UMI-UDI Primer Set, UMI Add-on Reagents, and purified with Clarefy Purification Beads (Claret Bioscience, CBS-K250B-24, CBS-UM-24, CBS-UR-24, CBS-BD-24). Since there is currently no optimized method to measure uscfDNA, 18 μL of extracted cfDNA was used as input and heat-shocked as instructed. To retain a high proportion of small fragments the low molecular weight retention protocol was followed for all bead-clean up steps. The index reaction PCR was run for 11 cycles. For double-stranded DNA libraries the NEB Ultra II (New England Bio, E7645S) was used with an 9 μL aliquot of extracted cfDNA according to the manufacturer's instructions with some modifications: the adapter ligation was performed using 2.5 μl of NEBNext® Multiplex Oligos for Illumina (Unique Dual Index UMI Adaptors RNA Set 1-NEB, cat #E7416S); the post-adapter ligation purification was performed using 50 μl of purification beads and 50 μl of purification beads' buffer, while the second (or post-PCR) purification was performed using 60 μl of purification beads (to retain smaller fragments). The PCR was performed using the MyTaq HS mix (Bioline, BIO-25045) for 10 PCR cycles.

### Sequencing.

Final library concentrations were measured using the Qubit Fluorometer (Thermo, Q33327) and quality assessed using the Tapestation 4200 using D1000 High-Sensitivity Tapes (Agilent, G2991BA and 5067-5584). Final libraries were sequenced on Illumina Novaseq 6000 instrument SP 300 flow cell type (2×150 bp).

### Bioinformatic Processing.

Sequence reads were demultiplexed using SRSLYumi (SRSLYumi 0.4 version, Claret Bioscience), python package. Fastq files were trimmed with (fastp, using adapter sequence (SEQ ID NO:12) AGATCGGAAGAGCACACGTCTGAACTCCAGTCA (r1) and (SEQ ID NO:13) AGATCGGAAGAGCGTCGTGTAGGGAAAGAGTGT (r2) and a Phred score of >15. Then sequenced reads were aligned against the combined human reference genome [GenBank: GCA_000001305.2] and LambdaPhage Genome [GeneBank GCA_000840245.1] using BWA-mem. broadinstitute.github.io/picard/. Samples were sorted and filtered using samtools (1.9 version). Reads were deduplicated by first moving the umi-tag using the bamtag tool from SRSLYumi (0.4 version), grouping with umi-tools (11.2 version), and removed using markduplicates from the Picard Toolkit (Quality control was performed with Qualimap (2.2.2c version). UMI-duplicate removal was done first by moving the UMI-tag with srslyumi-bamtag (SRSLYumi), marking with umi-tools (11.2 version), then removal with Picard (2.27.0 version). Bam files were split by size (uscfDNA 25-100 and mncfDNA 101-250) using alignmentSieve in deepTools (3.31 version). Correlation heatmaps were generated using bedGraph ToBigWig (version 4.0) and plotCorrelation in DeepTools (3.31 version). Functional peaks were first called with macs2 (2.2.7.1 version) and then analyzed with HOMERannotatePeaks (version 4.11.1).

### Nuclease Digestions for Analysis of Strandedness.

Prior to library preparation, the extracted cfDNA was digested with various strand-specific nucleases. For all reactions 500 μg of control oligos (350 nt ssDNA and 460 bp dsDNA lambda sequence, IDT) was spiked into 20 μL of extracted cfDNA. After the reaction, the DNA was purified by combining 30 μL of reaction buffer and 90 μL of SPRI-select beads, 90 μL of 100% isopropanol and incubated for 10 minutes. The tube was placed on a magnetic rack for five minutes to allow for the beads to migrate. The supernatant was discarded and the beads were washed twice with 200 μL of 80% ethanol. Once the second ethanol wash was removed the beads were left to air dry for 10 minutes. The beads were then resuspended in 20 μL of Qiagen elution buffer (or TrisHCl pH 8 10 mM).

Non-strand specific DNA digestion: 20 μL cfDNA was combined with 1 μL DNase I (Invitrogen, 18-068-015), 3 μL 10×DNase 1 Buffer, 6 μL of ddH₂O incubated for 15 minutes at 37° C. and heat inactivated for 15 minutes at 80° C. with 1 μL of 0.5M EDTA.

ssDNA-specific Digestion: 20 μL cfDNA was combined with 1 μL 1×S1 (Thermo, EN0321), 6 μL 5×S1 Buffer, 3 μL of ddH₂O incubated for 30 minutes at room temperature and heat inactivated for 15 minutes at 80° C. with 2 μL of 0.5M EDTA.

ssDNA-specific Digestion: 20 μL cfDNA was combined with 1 μL 0.1×P1 (NEB, M0660S), 3 μL NEBuffer r1.1, 6 μL of ddH₂O incubated for 30 minutes at 37° C. and inactivated with 2 μL of 0.5M EDTA.

ssDNA-specific Digestion: 20 μL cfDNA was combined with 3 μL Exonuclease 1 (NEB, M0293S), 3 μL 10×Exo 1 Buffer, 4 μL of ddH₂O incubated for 30 minutes at 37° C. and heat inactivated for 15 minutes at 80° C. with 1 μL of 0.5M EDTA.

dsDNA-specific Digestion: 20 μL cfDNA was combined with 2 μL dsDNase (ArcticZyme, 70600-201), 8 μL of ddH₂O incubated for 30 minutes at 37° C. and heat inactivated for 15 minutes at 65° C. with 1 mM DTT.

Nick Repair Analysis: 20 μL cfDNA was combined with 1 μL PrePCR Repair (NEB, M0309S), 5 μL ThermoPol Buffer (10×), 0.5 μL of NAD+ (100×), 2 μL of Takara 2.5 mM dNTP, 21.5 ddH₂O incubated for 30 minutes at 37° C. and placed on ice.

RNA Digestion: 20 μL of cfDNA was combined with 1 μL of RNase Cocktail (Thermo, AM228). For 20 minutes at 30° C. prior to input into the library preparation.

ssDNA Ladder to Determine Efficiency.

2 ng ssDNA ladder of various sizes (30-200) was spiked in 1 mL healthy plasma prior to extraction. Final elution was 40 μL and 18 μL was used for each final library. Oligonucleotides were manufactured by a commercial vendor (IDT, Custom Order).

### Scanning Electron Microscope (SEM).

After processing PBS or plasma samples with QiaC or QiaM protocol, the columns were air-dried at room temperature. They were cut into proper height to expose the membrane and fitted to the sample stage. The samples were coated with platinum and the detailed morphology of the membrane was examined by Focus-Ion Beam/Scanning Electron Microscopy (FEI, Nova 200 NanoLab).

### Quantification and Statistical Analysis.

Quantification of “% uscfDNA” was performed by calculating the ratio of the sample intensity (FU) of the electropherogram images between the ultrashort region (180-250 bp) and the mncfDNA (251-350 bp). Similarly, sample intensity was used to calculate the fold change of % Area cfDNA to control. A paired two-tailed student-test test was performed after ANOVA analysis in order to determine statistical significance. * p 0.05, ** p 0.01, and *** p 0.001. Bars graphs represent standard error of Mean (SEM).

The Experimental results are now described.

BRcfDNA-Seq can Purify and Visualize Ultrashort cfDNA in Plasma

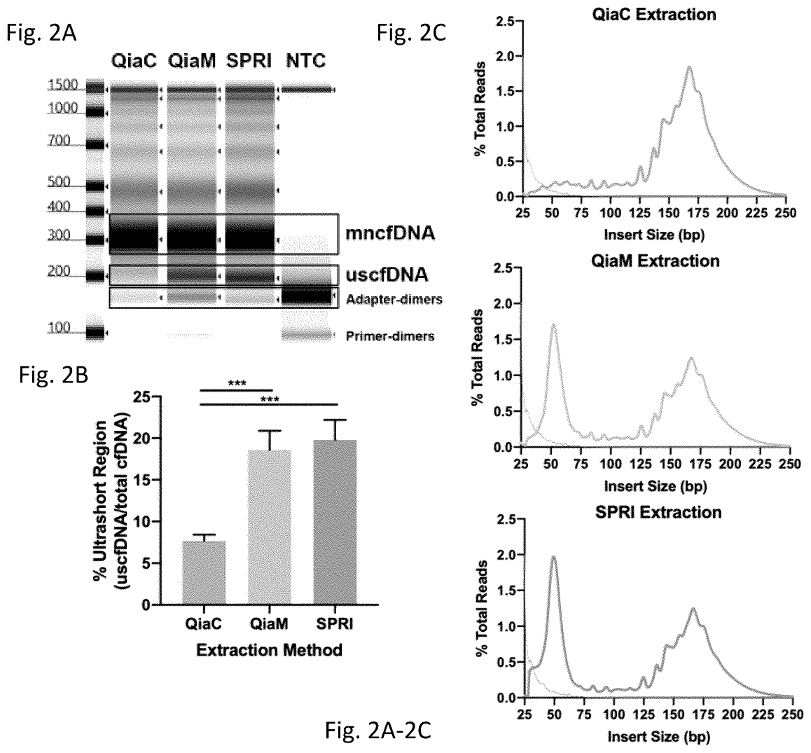

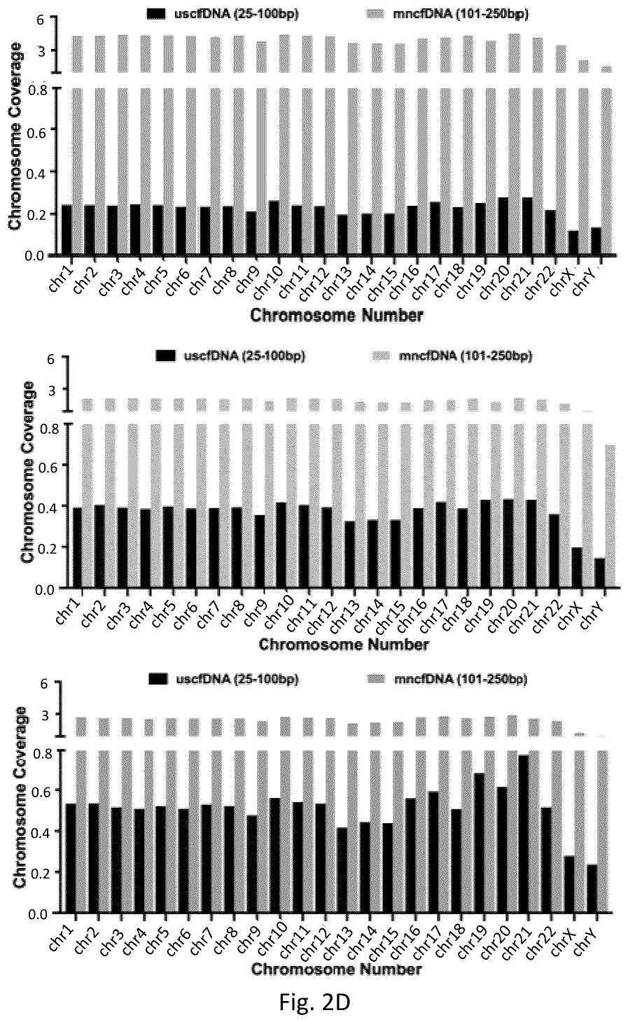

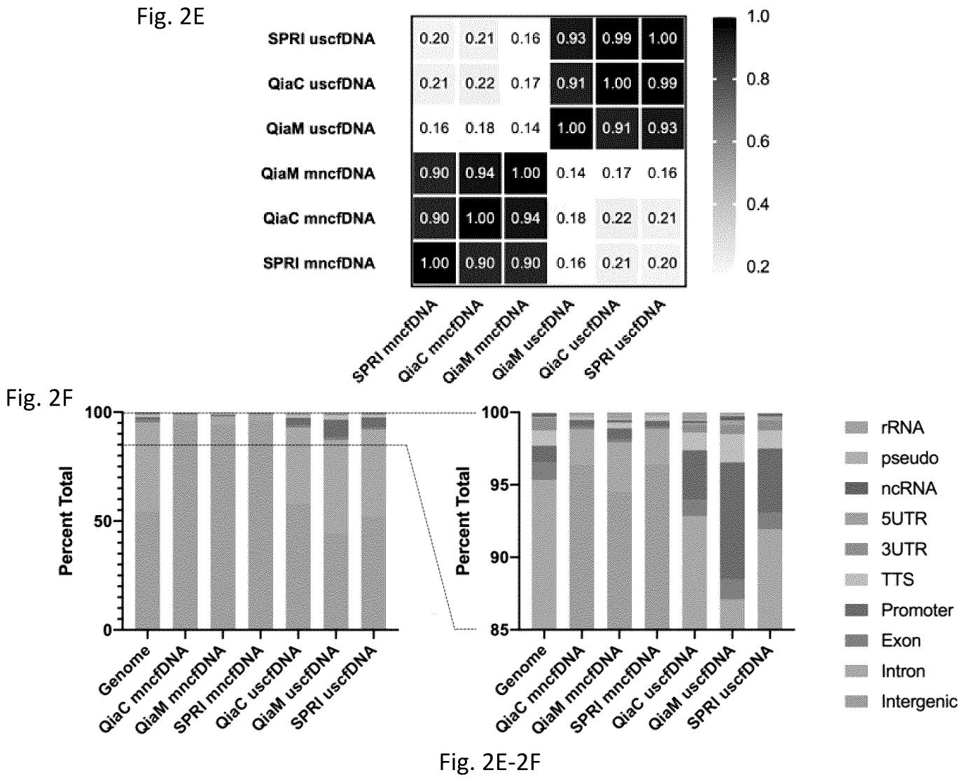

Single-stranded libraries (FIG. 1B) were made from cell-free DNA extracted by QiaM and SPRI methods which revealed a distinct cfDNA band at 200 bp in the electropherogram corresponding to about 50 bp of insert size (the library preparation adds about 150 bp-worth of adapters) compared to QiaC (FIGS. 2A and B). In all three extraction methods, the mncfDNA peak (300 bp before adapter removal) is present.

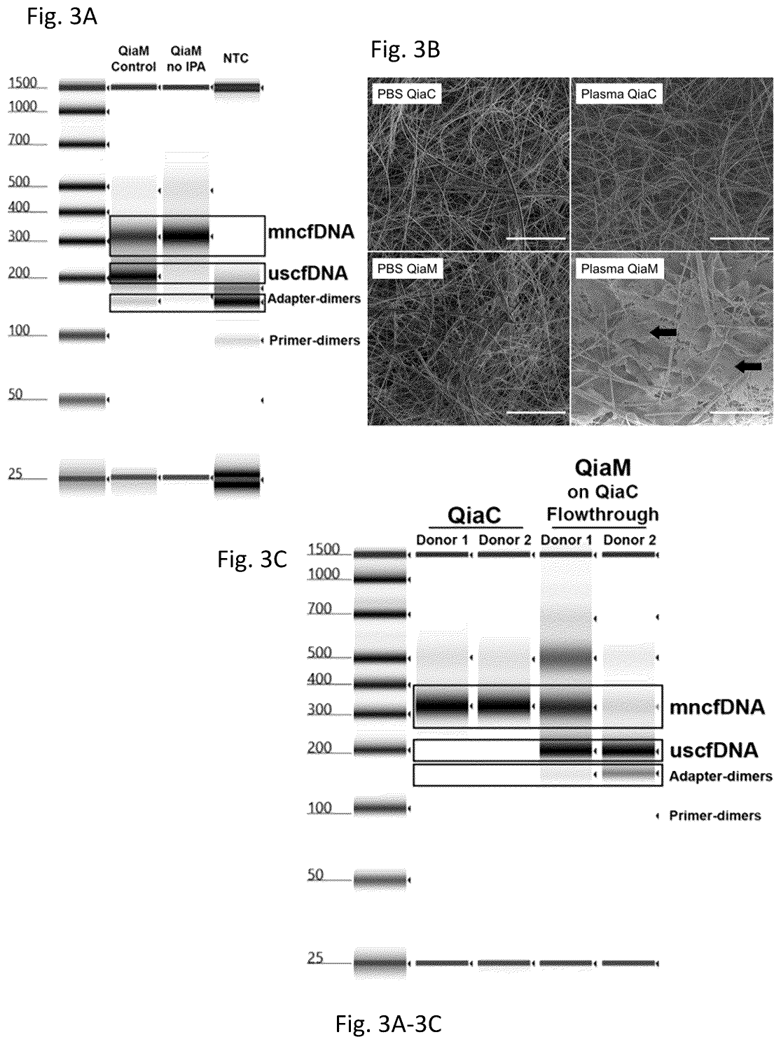

Similarly, using the QiaM which incorporates higher isopropanol volume enhanced the capture of low-molecular nucleic acids (FIG. 1A and FIG. 3A). Interestingly, the miRNA purification protocol is associated with slower flow through the silica column. SEM images of the silica column indicate a reduction in pore size accompanied by sheet-like deposits possibly derived from increased isopropanol precipitation of organic matter in the plasma (FIG. 3B). As part of BRcfDNA-Seq these two extraction methods optimized for short DNA are partnered with a single-stranded library construction in order to fully visualize and examine the cfDNA population that is smaller than 100 bp.

In a supplemental experiment, the QiaC protocol with centrifuge (as opposed to vacuum) was used in order to collect the flow through of the binding step of the standard QIaC protocol for the presence of low-molecular weight DNA. The QiaC flow through was subsequently extracted with QiaM (with increased isopropanol and lysis and binding buffers) to reveal that the uscfDNA could be rescued (FIG. 3C). This also indicates that the QiaC protocol has a tendency to lose low-molecular DNA.

uscfDNA is Consistently Present in Plasma Independent of Blood Collection Methods

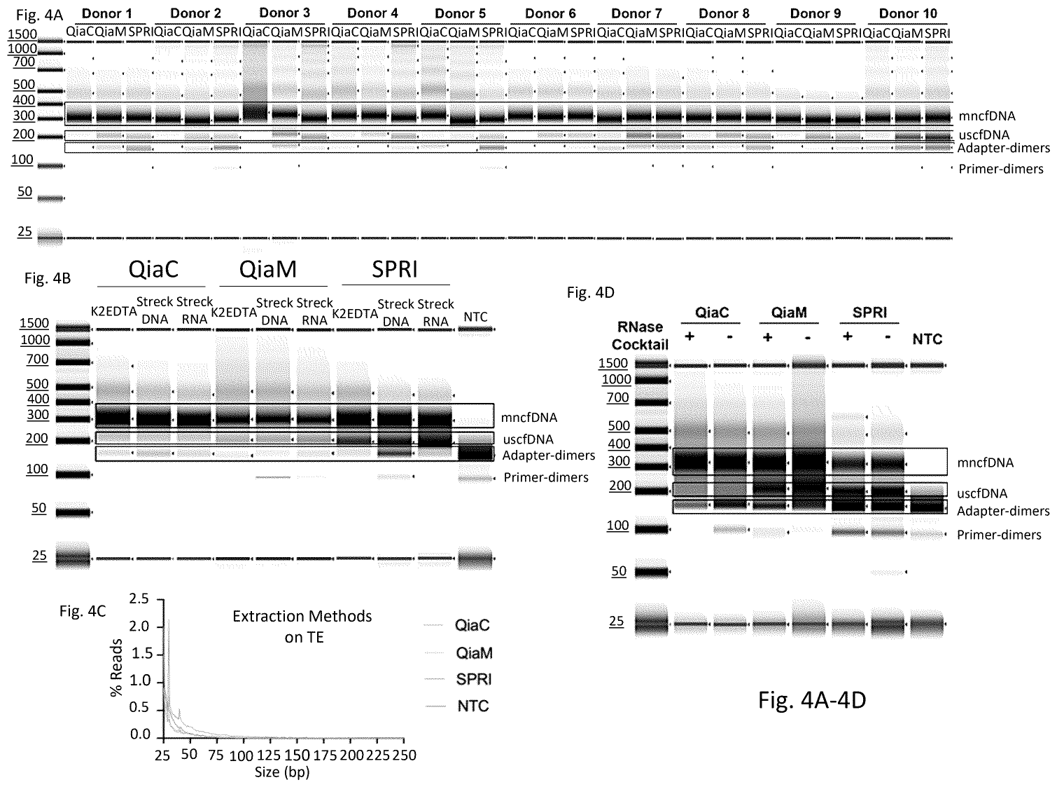

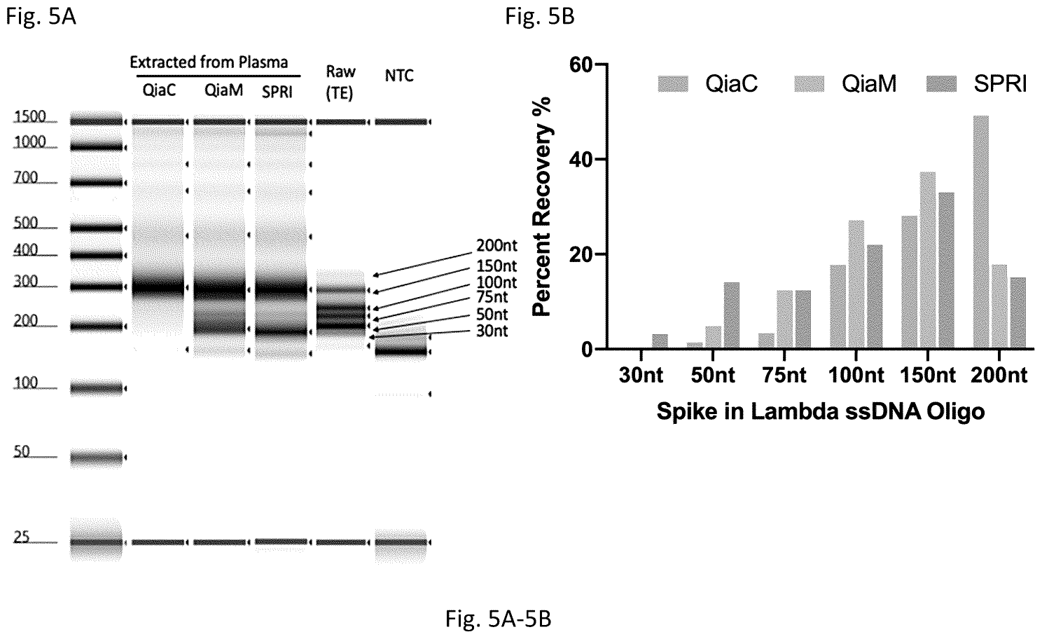

This is a reproducible phenomenon with similar observations in multiple donors (FIG. 2B and FIG. 4A). Although we have shown that plasma from K2EDTA vacu-containers contain uscfDNA (FIG. 2), K2EDTA tubes are often reported to be associated with cell-free DNA degradation (Parpart-Li et al., 2017, Clin. Cancer Res, 23:2471-2477). Thus, to rule out the possibility of uscfDNA as an artifact of sample collection, StreckDNA tubes (the gold-standard for cell-free DNA preservation due to their ability to decrease white blood cell rupture and subsequent genomic DNA contamination in the sample) was also tested for presence of uscfDNA. An alternative, StreckRNA, which is used to preserve RNA (a low molecular nucleic acid) and exosomes was also tested. All three collection tubes allowed us to detect the presence of the uscfDNA population (FIG. 4B). Extractions performed from the TE buffer alone did not manifest any uscfDNA or mncfDNA bands except for adapter-dimer bands introduced by the library preparation protocol (FIG. 4C). Additionally, treatment with RNase Cocktail digestion prior to library preparation did not appreciably decrease the uscfDNA band ruling out the presence of RNA.

Magnetic Bead Extraction Methods May Capture Short and Single-Stranded DNA Molecules Better than Silica Column-Based Methods

In order to compare the efficiency of the extraction methods, non-human ssDNA oligos designed from the E. coli phage lambda genome of sizes 30, 50, 75, 100, 150, and 200 nt (Table 2) were spiked into the plasma prior to extraction and library preparation. The uscfDNA extraction methods (QiaM and SPRI) retain ultrashort fragments in plasma with greater efficiency compared to the regular QiaC protocol (FIGS. 5A and B). Interestingly, the SPRI extraction method showed improved retention of 30 and 50 nt ssDNA compared to QiaM. Although these two extraction methods show improved ability in retaining low-molecular ssDNA, their yield suggests that there is still substantial loss. Hence, further refining of future methods to improve the yield is warranted. Advantages of the current bead-based methods is that they limit physical loss of ultrashort cfDNA fragments compared to silica columns that utilize flow through the pores. However, the observed presence of adapter-dimers is suggestive of the presence of inhibiting factors in SPRI derived cfDNA products that may interfere with downstream enzyme activity.

TABLE 2
Synthetic Oligomers and Primers

| | | | Lambda phage | |
|---|---|---|---|---|
| Name | Size | ss/ds | region | Notes |
| Lambda dsDNA | 459 bp | ds | 27′944:28′402 | PCR product, no UMI |
| Control | | | | |

SEQ ID NO: 1
5′-
CAAACTGCGCAACTCGTGAAAGGTAGGCGGATCCCCTTCGAAGGAAAGACCTGATGCTTTTCGTG
CGCGCATAAAATACCTTGATACTGTGCCGGATGAAAGCGGTTCGCGACGAGTAGATGCAATTATG
GTTTCTCCGCCAAGAATCTCTTTGCATTTATCAAGTGTTTCCTTCATTGATATTCCGAGAGCATCAAT
ATGCAATGCTGTTGGGATGGCAATTTTTACGCCTGTTTTGCTTTGCTCGACATAAAGATATCCATCT
ACGATATCAGACCACTTCATTTCGCATAAATCACCAACTCGTTGCCCGGTAACAACAGCCAGTTCC
ATTGCAAGTCTGAGCCAACATGGTGATGATTCTGCTGCTTGATAAATTTTCAGGTATTCGTCAGCC
GTAAGTCTTGATCTCCTTACCTCTGATTTTGCTGCGCGAGTGGCAGCGACATGGTTTGTTGT-3′

| | | | | |
|---|---|---|---|---|
| Lambda ssDNA | 350 nt | ss | 7′582:7′930 | IDT synthesized |
| Control | | | | |

SEQ ID NO: 2
5′-
CCTGGCCAGAATGCAATAACGGGAGGCGCTGTGGCTGATTTCGATAACCTGTTCGATGCTGCCAT
TGCCCGCGCCGATGAAACGATACGCGGGTACATGGGAACGTCAGCCACCATTACATCCGGTGAG
CAGTCAGGTGCGGTGATACGTGGTGTTTTTGATGACCCTGAAAATATCAGCTATGCCGGACAGGG
CGTGCGCGTTGAAGGCTCCAGCCCGTCCCTGTTTGTCCGGACTGATGAGGTGCGGCAGCTGCGG
CGTGGAGACACGCTGACCATCGGTGAGGAAAATTTCTGGGTAGATCGGGTTTCGCCGGATGATGG
CGGAAGTTGTCATCTCTGGCTTGGAC-3′

| | | | | |
|---|---|---|---|---|
| lambda 200 | 198 nt | ss | 12′051:12′248 | IDT synthesized, |
| | | | | internal-UMI 12 nt |

SEQ ID NO: 3
5′-
AAGGCGGAGAGTCAGTTCGCGGNNNNNNNNNNNNCGGCGCAACGTCGCCAGCTGTCTGCACAG
GAGAAATCCCTGCTGGCGCATAAAGATGAGACGCTGGAGTACAAACGCCAGCTGGCTGCACTTGG
CGACAAGGTTACGTATCAGGAGCGCCTGAACGCGCTGGCGCAGCAGGCGGATAAATTCGCACAG
CAGCAA-3′

| | | | | |
|---|---|---|---|---|
| lambda 150 | 150 nt | ss | 35′073:35′201 + | IDT synthesized, |
| | | | UMI | 3′-UMI 12 nt |

SEQ ID NO: 4
5′-
GCGTCCACTGCATGTTATGCCGCGTTCGCCAGGCTTGCTGTACCATGTGCGCTGATTCTTGCGCT
CAATACGTTGCAGGTTGCTTTCAATCTGTTTGTGGTATTCAGCCAGCACTGTAAGGTCTATCGGATT
TAGTGCNNNNNNNNNNNN-3′

| | | | | |
|---|---|---|---|---|
| lambda 100 | 100 nt | ss | 41′091:41′178 + | IDT synthesized, |
| | | | UMI | 3′-UMI 12 nt |

SEQ ID NO: 5
5′-
TCGTTAGTTTCTCCGGTGGCAGGACGTCAGCATATTTGCTCTGGCTAATGGAGCAAAAGCGACGG
GCAGGTAAAGACGTGCATTACGTNNNNNNNNNNNN-3′

| | | | | |
|---|---|---|---|---|
| lambda 75 | 75 nt | ss | 18′204:18′266 + | IDT synthesized, |
| | | | UMI | 3′-UMI 12 nt |

SEQ ID NO: 6
5′-
TCGTATCGCATTTATTGACCCGGCAAACGGGAATGAAACGCCGATGTTTGTGGCGCAGGGCAANN
NNNNNNNNNN-3′

| | | | | |
|---|---|---|---|---|
| lambda 50 | 50 nt | ss | 2′321:2′359 + | IDT synthesized, |
| | | | UMI | 3′-UMI 12 nt |

SEQ ID NO: 7
5′-ACCGCTTCCCGGTGCCGTTCACTTCCCGAATAACCCGGANNNNNNNNNNNN-3′

| | | | | |
|---|---|---|---|---|
| lambda 30 | 30 nt | ss | 4′278:4′300 + | IDT synthesized, |
| | | | UMI | 3′-UMI 9 nt |

SEQ ID NO: 8
5′-ACGCGGTGACGACTATCAGGAAANNNNNNN-3′

| | | |
|---|---|---|
| 17 Extension | 75 nt | SEQ ID NO: 9 |
| Primer Sequence | | 5′- |
| (i7 ext) | | CAAGCAGAAGACGGCATACGAGATNNNNNNNNNNNNNNNNNGTGA |
| | | CTGGAGTTCAGACGTGTGCTCTTCCGATCT-3′ |
| Forward Index | 70 nt | SEQ ID NO: 10 |
| Primer Sequence | | 5′- |
| (15) | | AATGATACGGCGACCACCGAGATCTACACNNNNNNNNACACTCTTT |
| | | CCCTACACGACGCTCTTCCGATCT-3′ |
| Reverse Index | 21 nt | SEQ ID NO: 11 |
| Primer Sequence | | 5′- CAAGCAGAAGACGGCATACGA-3′ |
| (Ui7) | | |

**N denotes barcode sequence*

uscfDNA Reads Map Evenly and Predominantly to Nuclear Human DNA Sequences

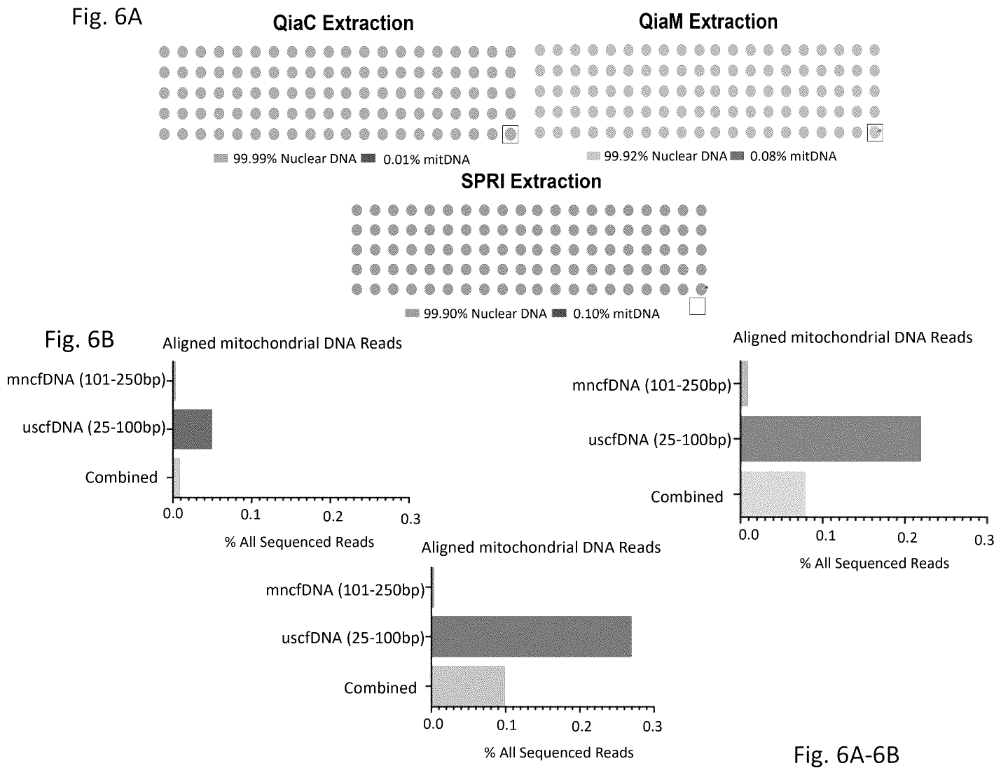

Upon sequencing and alignment to the human genome, the reads were divided into two distinct size populations (25-100 bp named uscfDNA and 101-250 bp named mncfDNA) with QiaM and SPRI both showing increased coverage of the ultrashort population (FIG. 2C). The reads corresponding to the ultrashort population are evenly distributed across the genome, although SPRI-extracted uscfDNA shows some increase in chromosomes 19 and 21 (FIG. 2D). It has been previously reported that mitochondria-derived cell-free DNA is fairly short (50 bp) but we found that it only contributed a minority (<0.1%) of the total mappable DNA reads (FIG. 6A). QiaM and SPRI are enriched for mitochondrial DNA in the uscfDNA population but still are a minor fraction of total DNA (FIG. 6B). Examining the correlation of the mapping between uscfDNA and mncfDNA extracted with the three methods revealed consistent homogeneity within the uscfDNA and mncfDNA populations (FIG. 2E).

The Functional Element Ratio of uscfDNA Sequences Resembles that of the Genome

The functional elements profile of the mncfDNA and uscfDNA sequences were examined amongst different extraction methods to identify any characteristic patterns (FIG. 2F). Compared to the genomic distribution of the functional elements, the mncfDNA profile presented an increased enrichment in the intergenic sequences and marked decrease in introns, exons, and promoters. In contrast, the uscfDNA more closely resembled the genome but had a noted increase in promoter, exon, and intron sequences. Between extraction methods, the QiaM-extracted uscfDNA had the greatest proportion of promoter regions mapping compared to QiaC and SPRI-extracted uscfDNA.

uscfDNA is Predominantly Single-Stranded

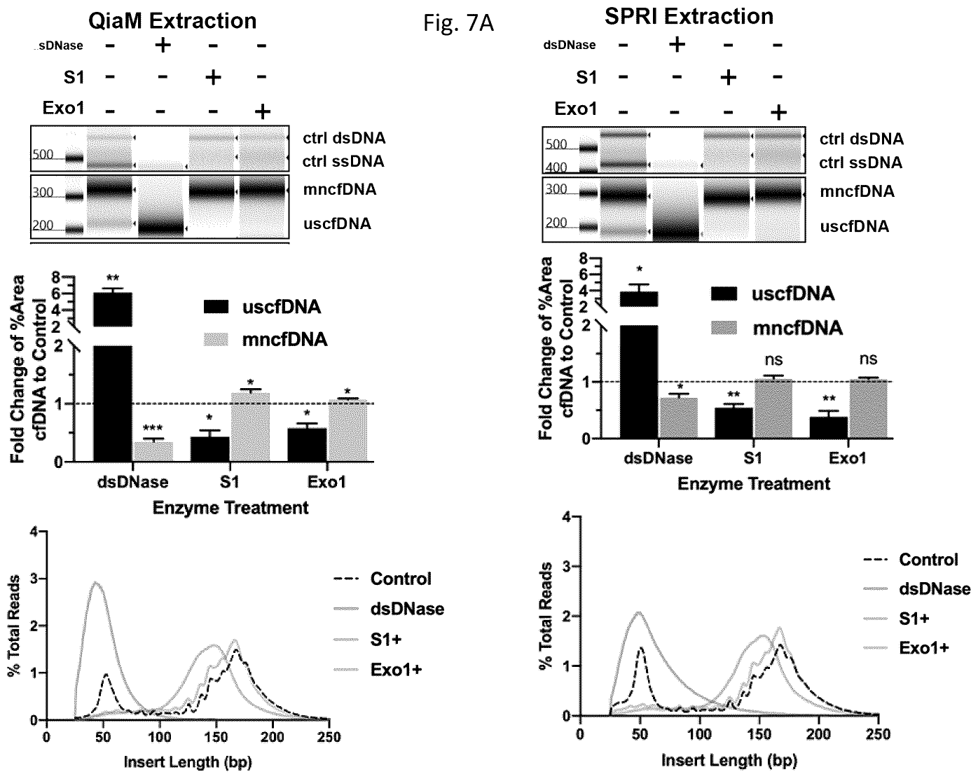

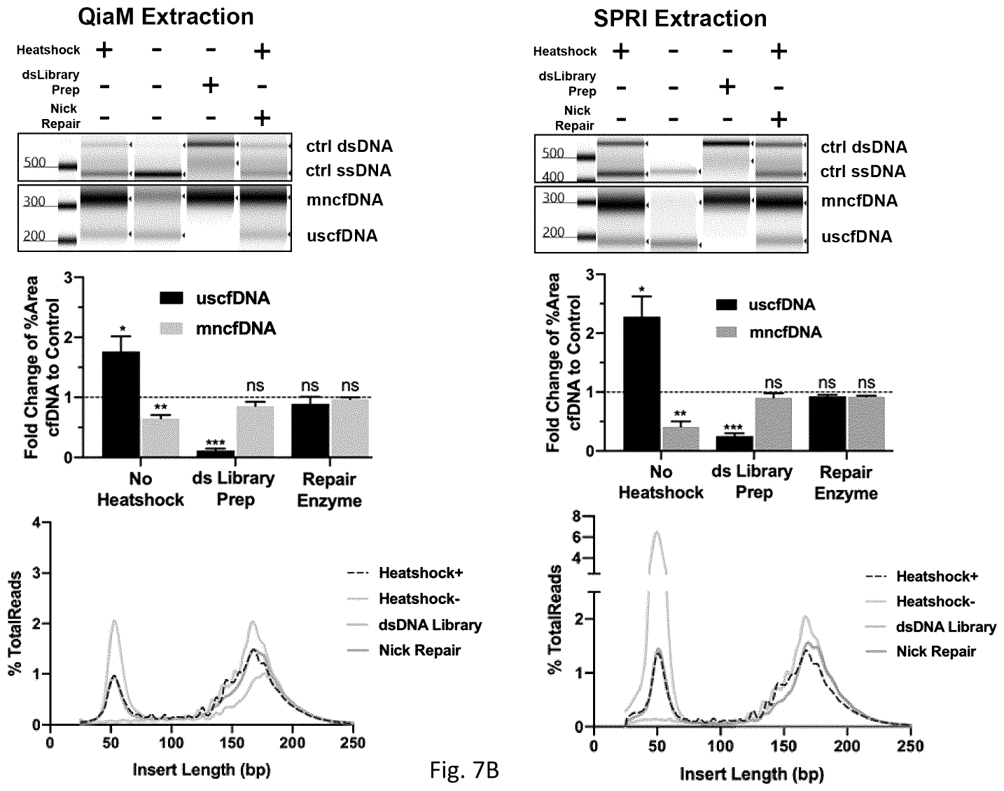

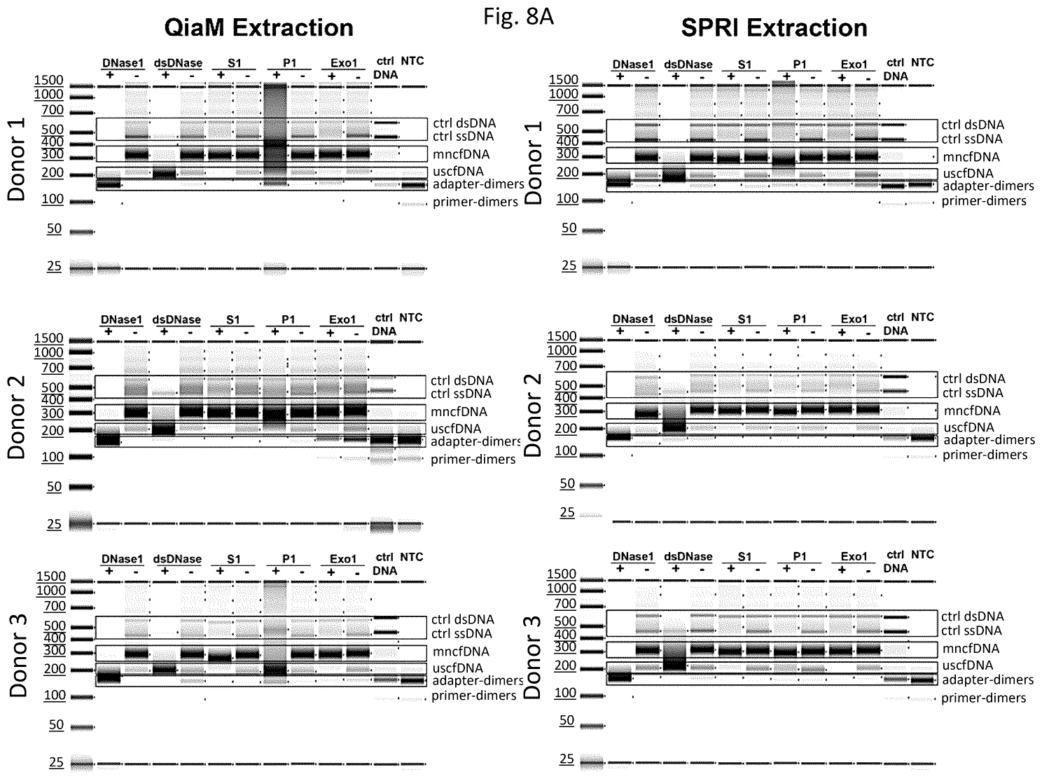

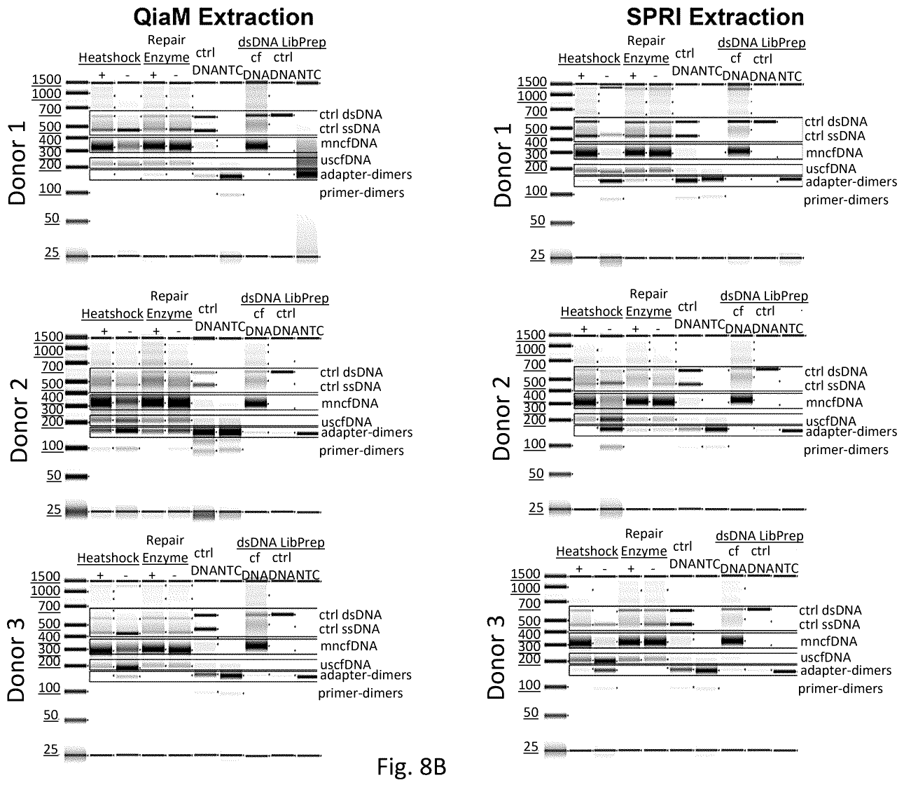

To examine the properties of strandedness, the extracted cfDNA supplemented with two control oligos (250 nt single-stranded and 350 bp double-stranded) was subject to strand-specific enzymes. When the DNA extracts were subject to dsDNA-specific DNase (dsDNase) digestion, the mncfDNA (300 bp) and the control dsDNA bands (500+bp) showed a clear reduction in intensity as evidenced by the electrophoresis of the corresponding final libraries (FIG. 7A and FIG. 8A). In contrast, digestion by single-strand specific nucleases (S1, Exo 1, and P1) showed significant reduction in the uscfDNA band and the control ssDNA band (400+bp) while preserving the mncfDNA band and the control dsDNA band (500+bp) in plasma extracted by both the QiaM and SPRI protocols. Sequencing and alignment of these libraries confirmed the results from the electropherograms (FIG. 7A, bottom panels). These results strongly indicate the single-stranded nature of the uscfDNA.

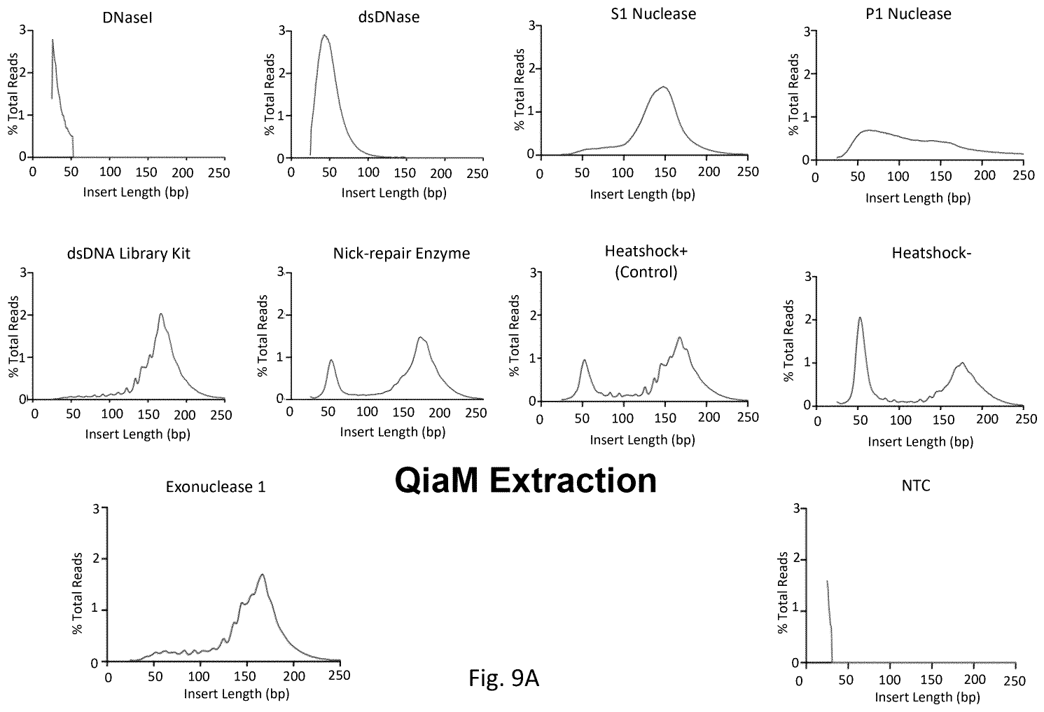

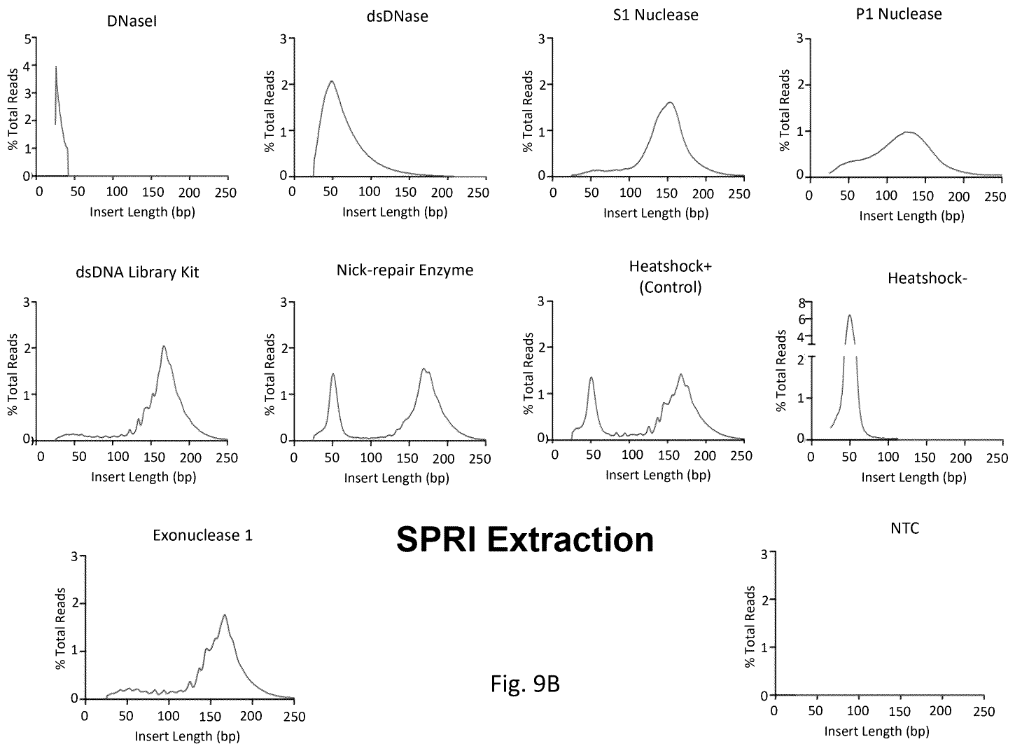

To corroborate the single-stranded nature of this DNA we leveraged the differences in the adapter ligation chemistry between ssDNA and dsDNA library kits (FIG. 7B). The uscfDNA peak was absent in the dsDNA library preparation (which only processes intact double-stranded substrates) suggesting that the ultrashort population is endogenously single-stranded in nature. By contrast, the ssDNA library kits require initial heat denaturation (98° C. for 3 minutes) to efficiently incorporate dsDNA molecules into the library. By skipping this step, the presence of the 200 bp population remained suggesting that the uscfDNA population is mostly single-stranded (FIG. 7B). Finally, to determine if the source of the uscfDNA derived from nicked dsDNA, we pre-treated the extracted nucleic acids with a nick repair enzyme but did not observe a reduction of ultrashort fragments in the final library. This suggests that the vast majority of uscfDNA are not derived from nicked mncfDNA. These observations were consistent among three replicates (FIGS. 8A and 9B).

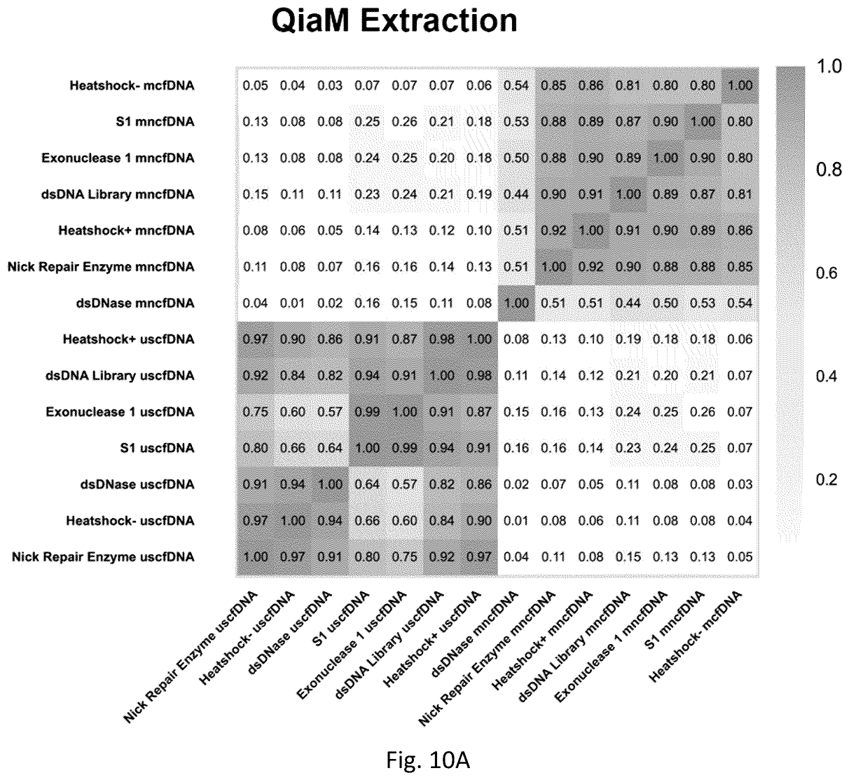

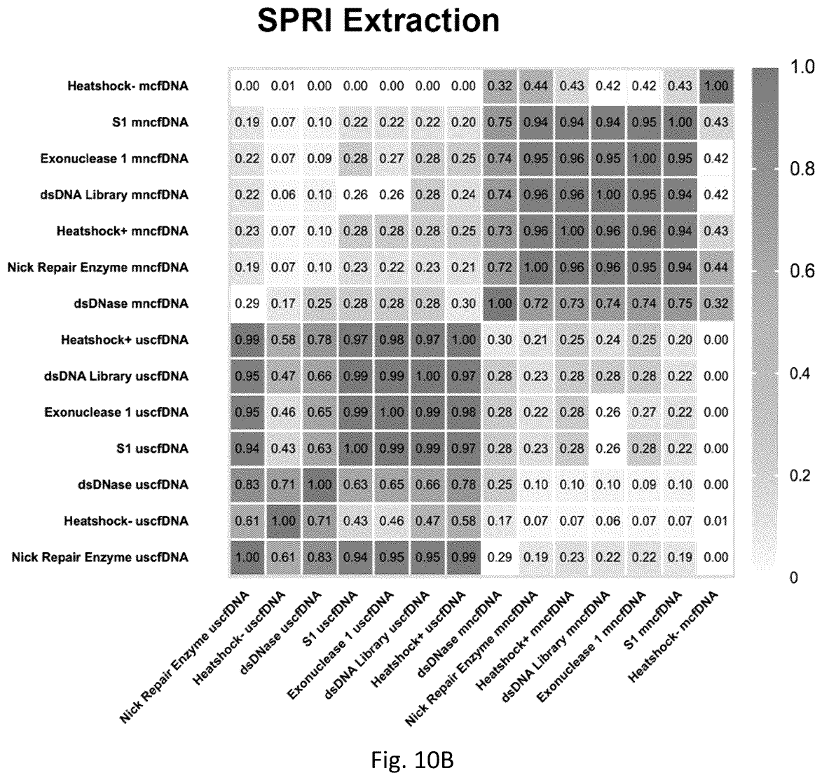

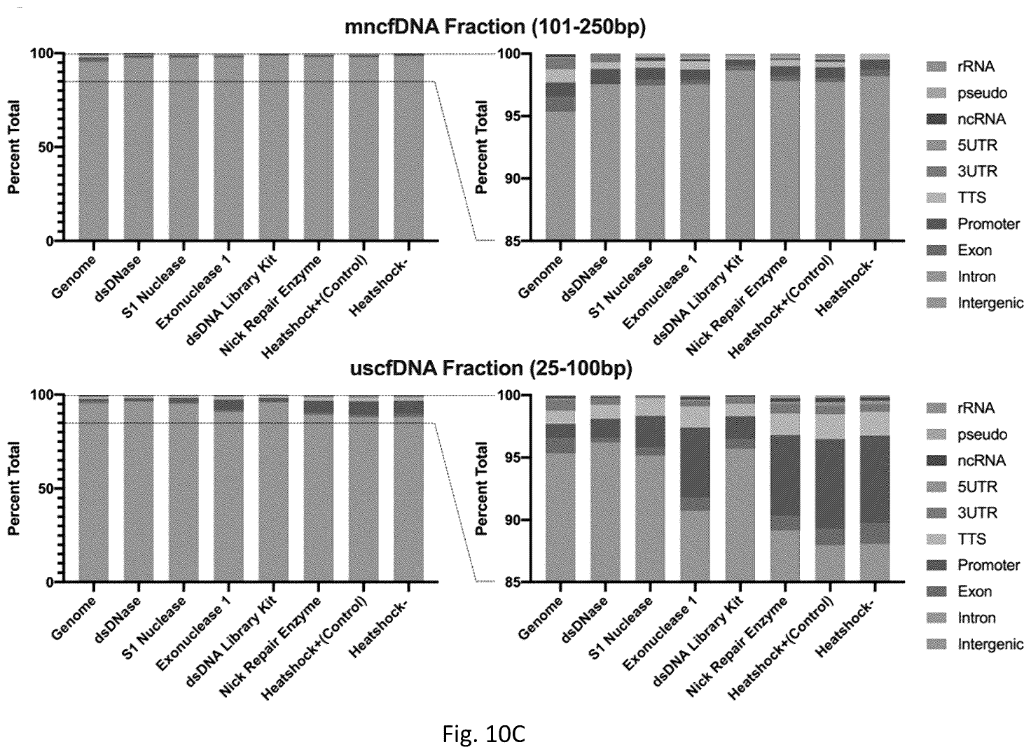

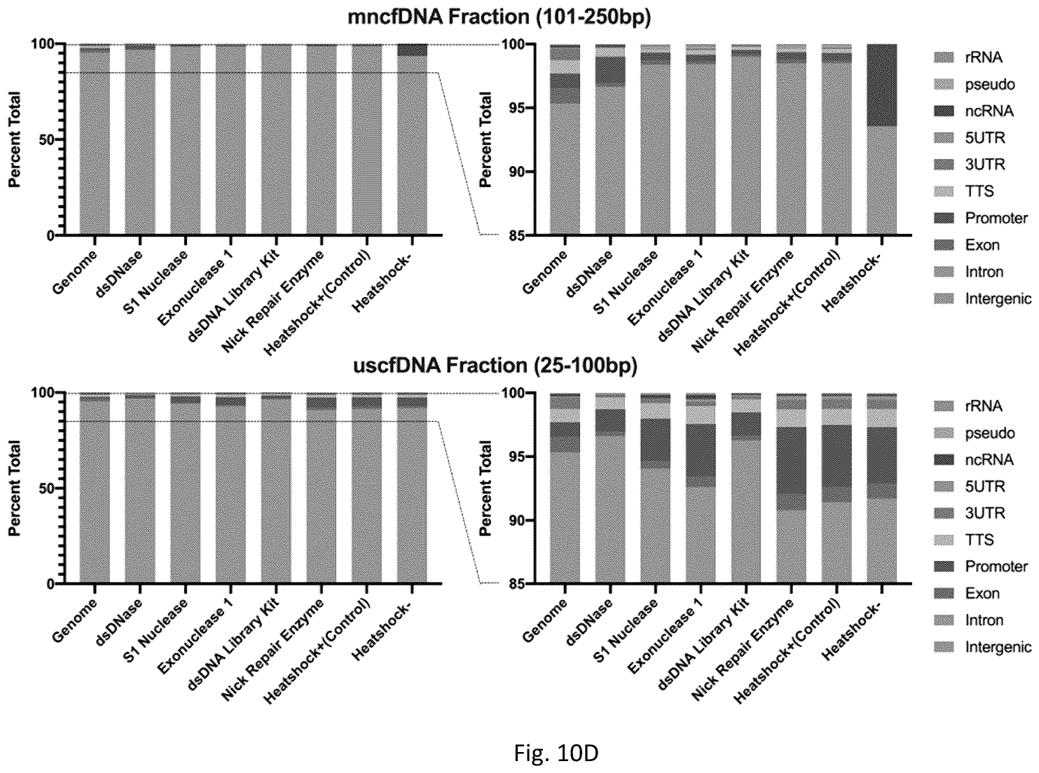

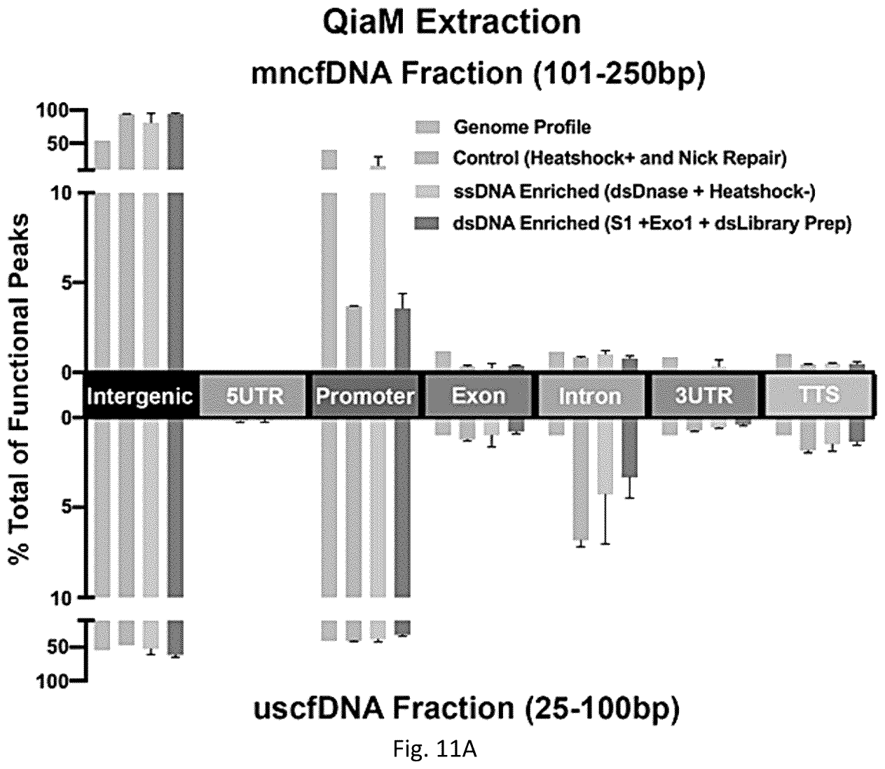

Alignment of sequenced digestion libraries recapitulated the findings previously mentioned with some interesting observations (FIGS. 7A, 7B and 9A and 9B). Firstly, the S1 treated samples showed a 10 bp downshift in the modality of the mncfDNA peak (from 160 to 150 bp). Secondly, both the S1 and nick-repair enzyme treatment flattened the periodicity on the left side of the mncfDNA peak. These observations suggest that the 10 bp periodicity may be a result of nicked mncfDNA at certain fragment lengths. The S1 enzyme may also be digesting jagged edges flanking the mncfDNA. Heatmap correlation of the digestions show that in both QiaM and SPRI extraction methods, the mncfDNA and uscfDNA populations group together (FIGS. 10A and 10B).

Functional Element Analysis of Digested Samples Corroborates with that uscfDNA has an Increased Proportion of Promoter, Intron, and Exon Regions Compared to Genome

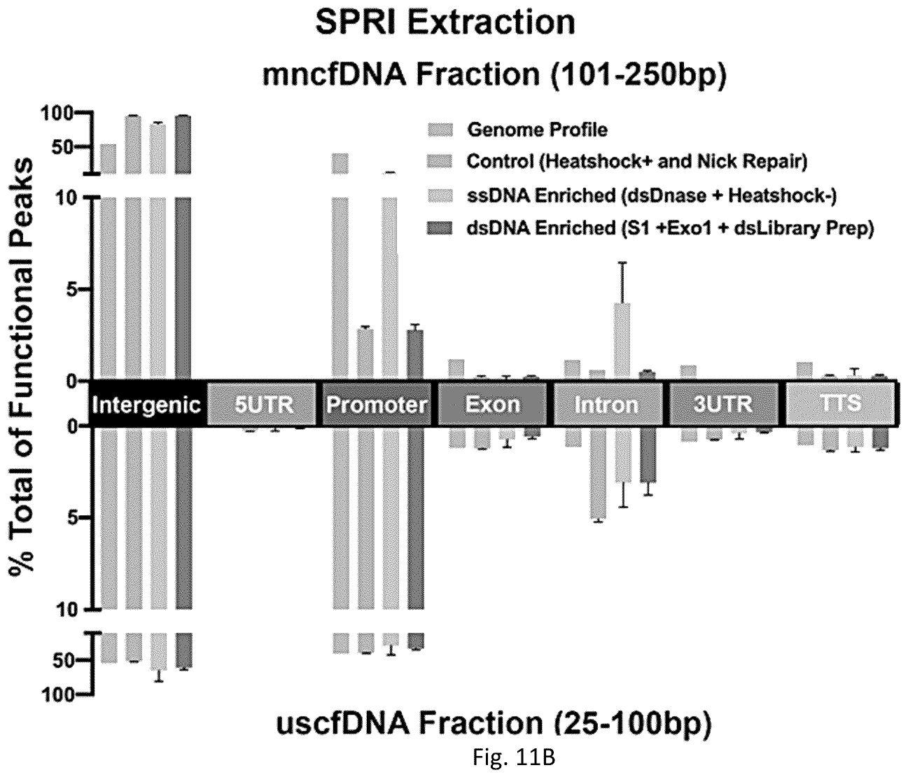

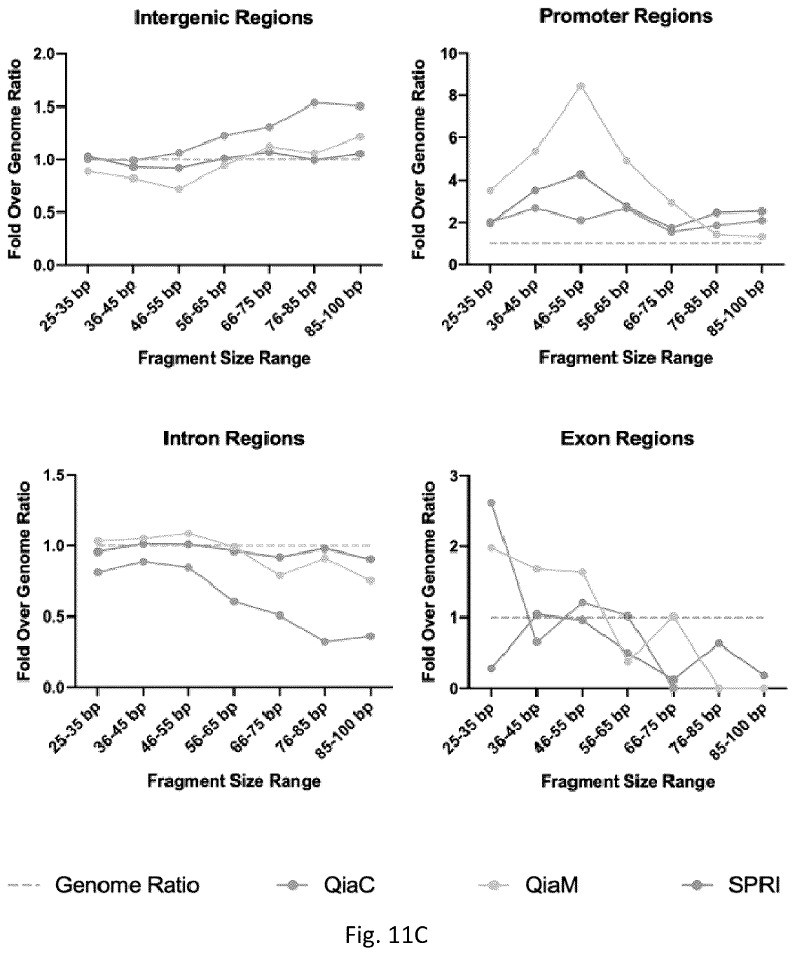

The functional element peak profiles (FIG. 10C, 10D) from the QiaM and SPRI digestions were used to see if they could generalize the functional characteristics differences in mncfDNA and uscfDNA observed earlier (FIG. 2F). By summating dsDNase and non-heat shock treatments to model uscfDNA enrichment and S1 nuclease, exo 1 nuclease, and dsDNA library preparation to model mncfDNA enrichment, we recipulated that uscfDNA is elevated in promoters, exons, and introns where mnfDNA is elevated in intergenic regions (FIG. 11A, 11B). Regardless, independent treatments revealed some unique findings. When samples were treated with dsDNase, the mncfDNA fraction appeared to mimic the uscfDNA (of untreated samples) in regards to increased promoter, exon, and intron fractions accompanied with a lowered intergenic localization. It initially appeared counter intuitive that dsDNase (which should reduce the mncfDNA) lead to a decrease in promoter and exon fraction in the uscfDNA fraction but it may be due degraded mncfDNA fragments flooding the uscfDNA size pool. Mirroring this, treatment with dsDNA library preparation led the uscfDNA fraction to mimic the mncfDNA by decreasing the promoter and exon ratio and increasing the intergenic regions.

The Proportion of Functional Peaks Vary at Different uscfDNA Fragment Sizes

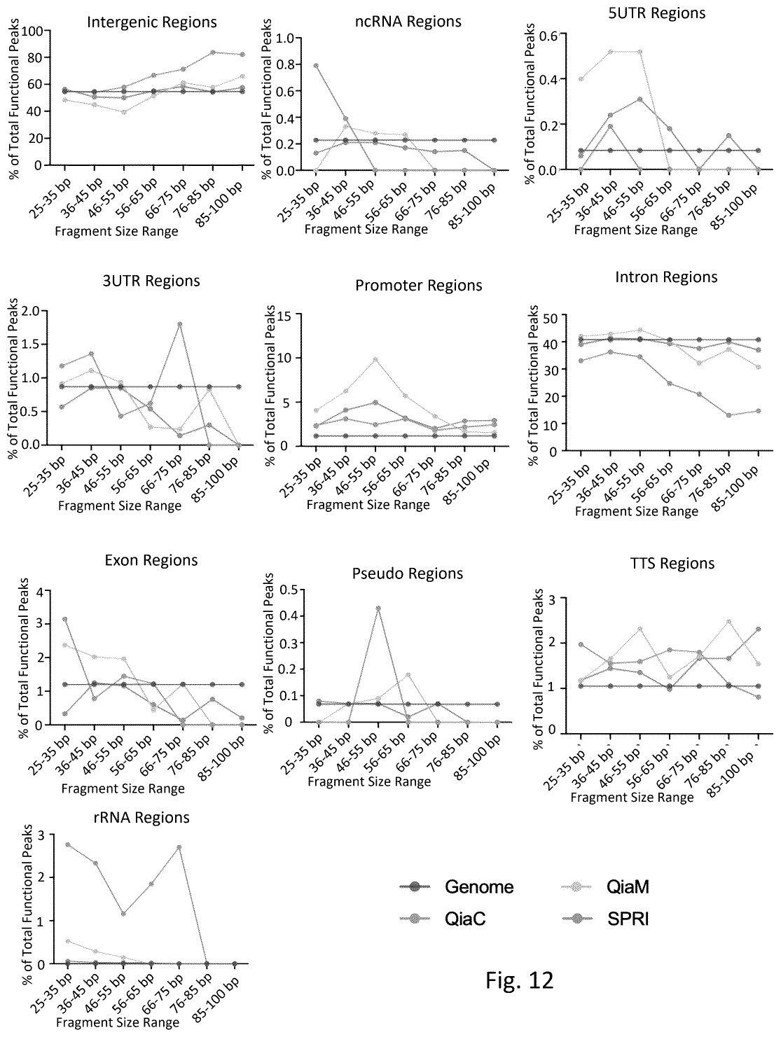

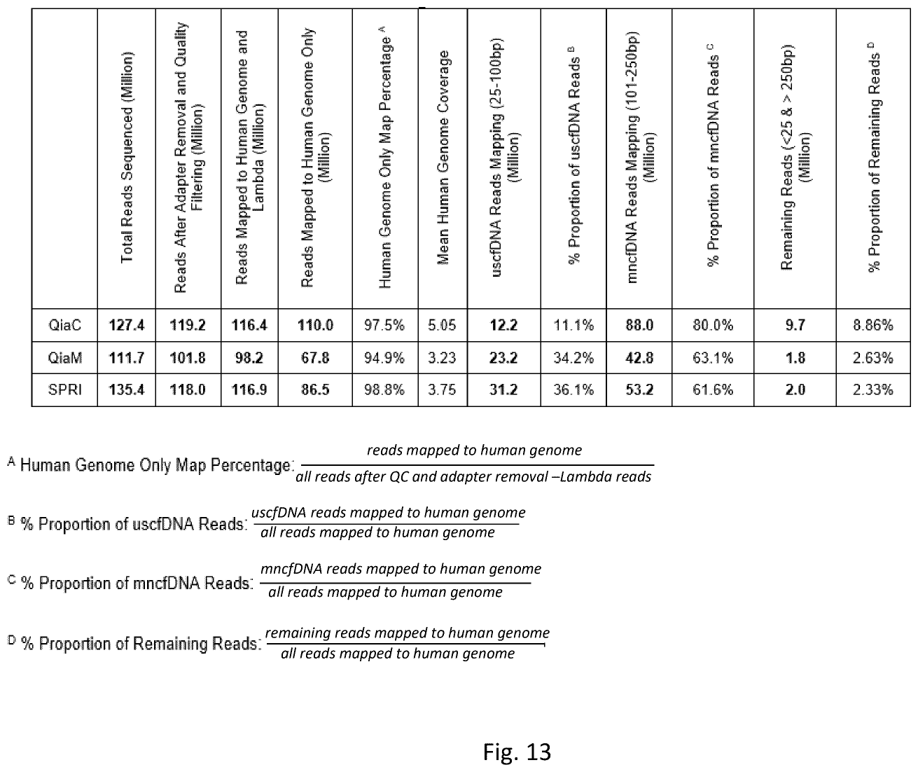

The uscfDNA population was divided in 10 bp-sized intervals to test whether there was an association between functional peak proportion and specific fragment sizes (FIGS. 11C and 12). In both QiaM and SPRI extraction methods there was a clear increase of promoter regions in sequences sized 45-55 bp compared to the genome and the QiaC extraction method. Similarly, a small increase occurred for introns and exons at 35-45 and 45-55 bp. Interestingly, the intergenic regions proportion increased steadily as the sequences got closer to 100 bp for all three extraction methods. Compared to QiaM and SPRI, QiaC behaved more sporadically due to having fewer total reads (43.4 vs 53.4 million) in the 25-100 bp region to begin with (FIG. 13).

### Example 2: Next-Generation Sequencing Pipeline to Detect Ultrashort Single-Stranded Cell-Free DNA

This invention is based in part on the development of a Next-generation Sequencing (NGS) pipeline to detect ultrashort single-stranded cell-free DNA (uscfDNA). This NGS pipeline unique in that it is able to detect and analyze ultrashort cell-free ssDNA of 25-75 bp in addition to the prototypical ˜150 bp mononucleosomal cfDNA (mncfDNA). This pipeline combines uscfDNA optimized extraction, ssDNA library construction with unique molecular identifiers, modified clean up-steps to preserve uscfDNA, and an established bioinformatic protocol (FIG. 14). Compared to dsDNA-NGS pipeline it is able to provide greater resolution of uscfDNA.

### Example 3: Ultrashort Single-Stranded Cell-Free DNA in Biofluids for Disease Detection

This invention encapsulates the detection and analysis of ultra-short single-stranded cell-free DNA (uscfDNA) in patient biofluids as a biomarker for disease. The uscfDNA may potentially contain existing somatic mutations or novel mutations useful for identifying cancer. uscfDNA may contain methylated markers that can be used to identify auto-immunity diseases. The uscfDNA may also be useful for as a global biomarker in which its increase concentration may be diagnostic of aberrations in the patient's condition.

### Example 4: Analysis of Ultrashort Single-Stranded Cell-Free DNA in Patient Saliva for Disease Detection

This invention encapsulates the detection and analysis of ultra-short single-stranded cell-free DNA (uscfDNA) in patient saliva as a biomarker for disease. The uscfDNA may potentially contain existing somatic mutations or novel mutations in the promoter regions useful for identifying cancer. uscfDNA may contain methylated markers that can be used to identify auto-immunity diseases. The uscfDNA may also be useful for as a global biomarker in which its increase concentration may be diagnostic of aberrations in the patient's condition.

1. A method of isolating ultrashort single-stranded cell-free DNA (uscfDNA) molecules from a sample, the method comprising the steps of:
a) contacting the sample with Solid Phase Reversible Immobilization (SPRI) magnetic beads to capture the uscfDNA;
b) contacting the sample with a mixture of phenol:chloroform:isoamyl alcohol to separate the uscfDNA away from contaminating proteins and peptides;
c) contacting the sample with Solid Phase Reversible Immobilization (SPRI) magnetic beads to clean up the uscfDNA; and
d) extraction of the uscfDNA.

2. The method of claim 1, further comprising the step of preparing a sequencing library from the extracted uscfDNA.

3. The method of claim 2, further comprising the step of sequencing the library of uscfDNA.

4. The method of claim 1, wherein the method further comprises a step of lysing a cell or disrupting proteins prior to step a).

5. The method of claim 4, wherein the step of lysing a cell or disrupting proteins comprises
i) adding Proteinase K and SDS to the sample,
ii) incubating the sample for 30 minutes at 60° C., and
iii) cooling the sample to ambient room temperature.

6. The method of claim 1, wherein step a) comprises:
i) adding SPRI magnetic size selection beads and isopropanol to the sample,
ii) incubating the sample at room temperature for at least 10 minutes,
iii) centrifuging the sample at 4000×G for at least five minutes,
iv) removing and discarding the supernatant, and
v) resuspending the pellet in buffer.

7. The method of claim 6, wherein step b) comprises:
i) aliquoting the resuspension solution from step a) v) into phase lock tubes,
ii) adding an equal volume (to the aliquot of the resuspension solution) of phenol:chloroform:isoamyl alcohol with equilibrium buffer,
iii) vortexing for at least 15 seconds,
iv) centrifuging the tubes at 19000×G for at least five minutes,
v) transferring the upper clear supernatant to a new tube; and
vi) repeating steps ii)-v) twice.

8. The method of claim 7, wherein step c) comprises performing at least two rounds of SPRI bead based clean up followed by ethanol precipitation.

9. The method of claim 1, wherein the sample is a biological fluid sample.

10. The method of claim 9, wherein the sample is selected from the group consisting of a blood sample, a plasma sample, a saliva sample, a sputum sample, a urine sample and a liquid biopsy sample.

11. A method of identifying novel biomarkers for diseases or disorders comprising obtaining uscfDNA from a sample according to the method of any one of claims 1-10 and analyzing the amount or sequence content of the uscfDNA to identify novel biomarkers of a disease or disorder.

12. The method of claim 11, wherein the biomarker is selected from the group consisting of a mutation, an indel, a copy number variation, and a methylation marker.

13. The method of claim 11, wherein the biomarker is an increase or decrease in the total amount of uscfDNA in a test sample as compared to a control sample.

14. The method of claim 11, wherein the biomarker is an increase or decrease in the amount of uscfDNA associated with a specific gene in a test sample as compared to a control sample.

15. A method of diagnosing a diseases or disorder in a subject in need thereof, the method comprising obtaining a sample from the subject, isolating uscfDNA from the sample according to the method of any one of claims 1-10; analyzing the amount or sequence content of the uscfDNA to detect a biomarker of a disease or disorder, and diagnosing the subject as having or at risk of the disease or disorder associated with the identified biomarker.

16. The method of claim 15, wherein the biomarker is selected from the group consisting of a mutation, an indel, a copy number variation, and a methylation marker.

17. The method of claim 15, wherein the biomarker is an increase or decrease in the total amount of uscfDNA in a test sample as compared to a control sample.

18. The method of claim 15, wherein the biomarker is an increase or decrease in the amount of uscfDNA associated with a specific gene in a test sample as compared to a control sample.

19. The method of claim 15, wherein the disease or disorder is selected from the group consisting of an autoimmune disease or disorder, a disease or disorder associated with an infectious agent, and cancer.

20. A kit comprising components for performing the method of any one of claims 1-10.
